(19) United States
(12) Patent Application Publication
(10) Pub. No.: US 2015/0238586 A1
(43) Pub. Date: Aug. 27, 2015

(54) Rapid Method Production High Purity Cancer Stem Cells and Population of High Purity Cancer Stem Cells

(71) Applicant: CALIFORNIA STEM CELL, INC., Irvine, CA (US)

(72) Inventors: Andrew Cornforth, Mission Viejo, CA (US); Michael McGary, Irvine, CA (US)

(21) Appl. No.: 14/421,413

(22) PCT Filed: Aug. 6, 2013
(86) PCT No.: PCT/US13/53850

§ 371 Date: Feb. 12, 2015

Related U.S. Application Data
(60) Provisional application No. 61/718,643, filed on Oct. 25, 2012.
(60) Provisional application No. 61/683,477, filed on Aug. 15, 2012.

(51) Int. Cl. A61K 39/00; A61K 35/13; A61K 35/15; C12N 5/095

(52) U.S. Cl. CPC A61K 39/0011; C12N 5/0695; A61K 35/13; A61K 35/15

(57) ABSTRACT

The disclosure provides reagents, including cells, and related methods, useful for administering to subjects with a neoplastic disorder. The reagents and methods encompass cancer stem cells of enhanced purity. Neoplastic disorder encompasses melanoma, ovarian cancer, colorectal cancer, breast cancer, and lung cancer.

### PRIORITY BENEFIT

The present application claims priority benefit from U.S. Provisional Ser. No. 61/718,643, filed Oct. 25, 2012, entitled, “Rapid Production of High Purity Cancer Stem Cells and Population of High Purity Cancer Stem Cells,” which is hereby incorporated herein in its entirety, and from U.S. Provisional Ser. No. 61/683,477, filed Aug. 15, 2012, entitled, “Rapid Method to Produce High Purity Cancer Stem Cells and Population of High Purity Cancer Stem Cells, which is also hereby incorporated herein in its entirety.

### FIELD OF THE DISCLOSURE

The present disclosure relates to cancer stem cells, methods and reagents for cell purification, methods for stimulating immune response, and methods for administration to subjects. The compositions and related methods can stimulate immune response against antigens that are characteristic of neoplastic disorders, or against cells that express the antigens. Neoplastic disorders of the present disclosure include melanoma, liver cancer, gastric cancer, and ovarian cancer.

### BACKGROUND

In a solid tumor, a small percentage of the cells have the capacity to initiate tumors of the same histological heterogeneity as the parental tumor. These cells are called, “cancer stem cells.” These are also known as tumor-initiating cells or cancer-initiating cells. Cancer stem cells can be defined by a cluster of properties. First, they have the capacity to renew themselves. Second, they are able to establish new tumors when transplanted. Third, they may be characterized as dormant or slowly cycling (cell cycle) tumor cells. Fourth, they may be responsible for resistance of tumors to chemotherapy or radiation therapy. Fifth, they depend on a particular microenvironment that maintains their ability to renew, and to give rise to more differentiated progenitor cells, where the environment maintains the undifferentiated state of the cancer stem cells. This microenvironment may include mesenchymal stem cells, tissue-associated fibroblasts, and endothelial cells. In the case of colon cancer stem cells, for example, this microenvironment includes the presence of tumor-associated myofibroblasts. (Schmidt et al (2011) Oncotarget. 2:313-320; Borovski et al (2011) Cancer Res. 71:634-639; Korkaya et al (2011) J. Clin. Inv. 121:3804-3809). The ability to form spheres with in vitro culture, is yet another characteristic that can contribute to the identification of a particular cell as a cancer stem cell (Perego et al (2011) J. Inv. Dermatol. 11:546-547). One non-limiting definition of cancer stem cells is, cells that are able to reproduce the full heterogeneity of the parental tumor and to grow continuously even after multiple passages (Civenni et al (2011) Cancer Res. 71:3098-3109).

Cancer stem cells have been shown to inhibit immune response, where the inhibitory mechanisms included induction of T regulatory cells (Tregs), an impairment of T cell activation and proliferation (Wei et al (2010) Clin. Cancer Res. 16:461-473).

Cancer stem cells establish and maintain tumor masses by their ability to continuously self-renew. In addition, tumor stem cells also migrate in what is called an epithelial-to-mesenchymal transition state. These features of self-renewal and migratory or invasive characteristics are believed to be the main reasons for cancer's virulence (Greaves et al (2012) Clonal evolution in cancer. Nature. 481(7381): p. 306-13). In addition, cancer stem cells have immunosuppressive properties (Wu et al (2010) Glioma cancer stem cells induce immunosuppressive macrophages/microglia. Neuro. Oncol. 12:1113-1125). Thus, cancer stem cells have been explored as a target for anti-cancer therapy, for example, by reagents and methods that destroy the cancer stem cells.

Putative tumor stem cells have been identified in a number of solid tumors based on markers and serial transplantation xenograph assays performed in mice. Several surface markers can identify tumor stem cells in melanoma but the expression of these markers is variable from tumor to tumor when assayed after surgical section. The biomarker CD271, is a growth factor receptor associated with cells of neural crest origin. CD271 can be used to identify putative melanoma stem cells, where these melanoma stem cells may be propagated in a mouse model under serial dilution (Civenni et al (2011) Human CD271-positive melanoma stem cells associated with metastasis establish tumor heterogeneity and long-term growth. Cancer Res. 71:3098-3109).

A characteristic of cells of the neural crest during embryonic development is their ability to migrate, a characteristic of mesenchymal cells. Melanoma cells that retain mesenchymal characteristics are an aggressive species of melanoma cell. CD146, also known as melanoma cell adhesion molecule (MCAM) and MUC18, is a marker of melanoma progression (Schlagbauer-Wadl et al (1999) Influence of MUC18/MCAM/CD146 expression on human melanoma growth and metastasis in SCID mice. Int J Cancer. 81:951-955). CD146 (MCAM) is also expressed by normal mesenchymal stem cells (Rusell et al (2010) Stem Cells. 28:788-798). The co-expression of these two markers on the same cell indicates a very aggressive form of cancer stem cell.

Traditional approaches using non-cancer stem cell specific media have been labor intensive and lengthy, with an average production time of 3.8 months (range 0.6 to 22.3 months, median 3.1). This resulted in delayed time to treatment with only 29% of the patients who submitted a sample receiving therapy. Frequently, overgrowth of normal fibroblast required extensive manipulation by skilled technicians which made the process expensive. Bulk preparations lack large amounts of antigen from the most aggressive phenotypes, namely tumor initiating or cancer stem cells.

By isolating and propagating putative cancer stem cells from patient tumor samples to quantities necessary for loading dendritic cells the present disclosure provides benefits beyond the traditional approach.

### SUMMARY OF THE DISCLOSURE

The present disclosure provides reagents, including cells, and related methods, useful for administering to subjects with a neoplastic disorder. The reagents and methods encompass cancer stem cells of enhanced purity. Neoplastic disorder encompasses melanoma, ovarian cancer, colorectal cancer, breast cancer, and lung cancer.

The present disclosure provides an isolated population of cells originating from a human melanoma tumor, wherein: (i) at least 30% of the cells in the population express CD146 and at least 30% of the cells in the population express CD271, or (ii) wherein at least 30% of the cells co-express CD146 and CD271, wherein the percent value (%) is defined as an average value over the population. Also, what is provided is the above isolated population of cells, wherein: the expression is at least 35%; and, co-expression is at least 35%. Also, what is provided is the above population of cells, wherein: the expression is at least 40%; and co-expression is at least 40%. Also, what is provided is the above population of cells, wherein: the expression is at least 45%; and, co-expression is at least 45%. In another aspect, what is provided is the above population of cells, wherein: the expression is at least 50%; and, co-expression is at least 50%.

What is also contemplated is the above population of isolated cells, wherein less than 5% of the cells are contaminating cells, or wherein less than 2% of the cells are contaminating cells.

In vaccine embodiments, what is provided is a vaccine comprising autologous dendritic cells, wherein the dendritic cells are loaded with the above isolated population of cells of, and wherein the dendritic cells and the human tumor are from the same human subject.

What is provided is the above vaccine, wherein the population of cells, prior to loading on the dendritic cells, comprises radiation damage that prevents cell division, or comprises a nucleic acid cross-linking agent that prevents cell division.

In another vaccine embodiment, what is provided is a vaccine comprising autologous dendritic cells, wherein the dendritic cells are loaded with at least one of the isolated population of cells originating from a human melanoma tumor, wherein: (i) at least 50% of the cells in the population express CD146 and at least 50% of the cells in the population express CD271, or (ii) wherein at least 50% of the cells co-express CD146 and CD271, wherein the percent value (%) is defined as an average value over the population, and wherein the dendritic cells and the human tumor are from the same human subject.

What is provided is the above vaccine, wherein the population of cells, prior to loading on the dendritic cells, comprises radiation damage that prevents cell division, or comprises a nucleic acid cross-linking agent that prevents cell division.

What is provided is an isolated population of cells originating from a human melanoma tumor, wherein at least 30% of the cells in the population express CD146 and at least 30% of the cells express CD271, or wherein at least 30% of the cells co-express CD146 and CD271, wherein the cells are prepared by a method comprising the steps of: Step i. Dispersing cells in a melanoma tumor sample, Step ii. Culture on a low adherent surface or on an ultra-low adherent surface, Step iii. Sedimentation to collect microspheres; and, Step iv. Dissociating cells from the microspheres.

What is further provided is the above method, further comprising the step (Step v.) of culturing in a culture medium on an adherent surface in order to expand cells, to produce a population of expanded cells.

What provided is the above method, wherein Step (ii) comprises culture on a low adherent surface, or wherein Step (ii) does not comprise culture on a low adherent surface, or wherein Step (ii) comprises culture on an ultra-low adherent surface, or wherein Step (ii) comprises culture on an ultra-low adherent surface and not on a low adherent surface.

What is provided is an isolated population of cells originating from a human melanoma tumor, wherein at least 30% of the cells in the population express CD146 and at least 30% of the cells express CD271, or wherein at least 30% of the cells co-express CD146 and CD271, wherein the cells are prepared by a method comprising the steps of: Step i. Dispersing cells in a melanoma tumor sample, Step ii. Culture on a low adherent surface or on an ultra-low adherent surface, Step iii. Sedimentation to collect microspheres; and, Step iv. Dissociating cells from the microspheres.

What is provided are the above population of cells, wherein the isolated population of cells has at least one of: (i) down-regulated immunosuppressive molecule; (ii) up-regulated MHC-II; or (iii) down-regulated immunosuppressive molecule and up-regulated of MHC-II; as compared with expression that is detectable in the cells in Step i.

What is provided are the above cells, wherein the immunosuppressive molecule is at least one of indoleamine-pyrrole-2,3-dioxygenase, tumor growth factor-beta, and interleukin-10 (IL-10), and wherein the down-regulation is to a level that is 80% or lower, as compared with expression (defined as 100%) that is detectable in Step i.

What is provided are the above cells, wherein the dispersing cells from one or both of the melanoma tumor sample and from the microspheres, comprises treatment with an added protease.

What is provided are the above cells, wherein culture on a low adherent surface is in the presence of basic fibroblast growth factor (bFGF).

What is provided is the above cells, wherein the culturing on a low adherent surface or ultra-low adherent surface comprises collecting any tumor stem cell spheres that have formed, wherein the collecting is performed every 2-3 days, with resumed culturing of the collected spheres in fresh medium on the low adherent surface.

In vaccine embodiments, what is provided is a vaccine comprising autologous dendritic cells, loaded with the isolated population of cells, as disclosed above, wherein the dendritic cells and the human tumor are from the same human subject.

In other vaccine embodiments, what is provided is the above vaccine, wherein tumor cell division is prevented, prior to loading on dendritic cells, by irradiating the tumor cells or by adding a nucleic acid cross-linking agent to the tumor cells.

What is provided is an isolated population of cells originating from a human melanoma tumor, wherein at least 30% of the cells in the population express CD146 and at least 30% of the cells express CD271, or wherein at least 30% of the cells co-express CD146 and CD271, wherein the cells are prepared by a method comprising the steps of: Step i. Dispersing cells in a melanoma tumor sample, Step ii. Culture on a low adherent surface or ultra-low adherent surface, Step iii. Sedimentation to collect microspheres; and, Step iv. Dissociating cells from the microspheres, and Step v. Culturing in a culture medium on an adherent surface in order to expand cells, to produce a population of expanded cells.

What provided is the above method, wherein Step (ii) comprises culture on a low adherent surface, or wherein Step (ii) does not comprise culture on a low adherent surface, or wherein Step (ii) comprises culture on an ultra-low adherent surface, or wherein Step (ii) comprises culture on an ultra-low adherent surface and not on a low adherent surface.

What is provided are the above cells, wherein the isolated population of cells has at least one of: (i) down-regulated immunosuppressive molecule; (ii) up-regulated MHC-II; or (iii) down-regulated immunosuppressive molecule and up-regulated of MHC-II; as compared with expression that is detectable in the cells in Step i.

What is provided are the above cells, wherein the immunosuppressive molecule is at least one of indoleamine-pyrrole-2,3-dioxygenase, tumor growth factor-beta, and interleukin-10 (IL-10), and wherein the down-regulation is to a level that is 80% or lower, as compared with expression (defined as 100%) that is detectable in Step i.

What is provided are the above cells, wherein the dispersing cells from one or both of the melanoma tumor sample and from the microspheres, comprises treatment with an added protease.

What is provided are the above cells, wherein culture on a low adherent surface is in the presence of basic fibroblast growth factor (bFGF).

What is provided are the above cells wherein culturing on an adherent surface in order to expand cells is in a culture medium that contains bFGF.

What is provided is the above cells, wherein the culturing on a low adherent surface comprises collecting any tumor stem cell spheres that have formed, wherein the collecting is performed every 2-3 days, with resumed culturing of the collected spheres in fresh medium on the low adherent surface.

What is provided is the above cells, wherein the total time of culturing on the adherent surface is selected from a time frame that is 12-30 days, 14-28 days, or 18-24 days.

In vaccine embodiments, what is provided is a vaccine comprising autologous dendritic cells, loaded with the isolated population of cells, as disclosed above, wherein the dendritic cells and the human tumor are from the same human subject.

In other vaccine embodiments, what is provided is the above vaccine, wherein tumor cell division is prevented, prior to loading on dendritic cells, by irradiating the tumor cells or by adding a nucleic acid cross-linking agent to the tumor cells.

In methods embodiments, what is provided is a method for stimulating an antigen-specific immune response against one or more melanoma-specific antigens, comprising administering to a human subject comprising living melanoma cells, a vaccine comprising autologous dendritic cells that are loaded with the above isolated population of cells of, wherein the dendritic cells and the human tumor are from the same human subject. What is also provided is the above method, wherein the melanoma-specific antigen is MAGE antigen.

In another methods embodiment, what is provided is a method for producing purified cancer stem cells, comprising the steps of: (a) immersing a cell suspension, previously acquired by dissociating cells of a tumor sample, in neuron stem cell media and culturing in ultra-low adherent container or in low adherent container; (b) allowing formation of cancer stem cell spheres; (c) recovering the cancer stem cell spheres by sedimentation to produce recovered spheres; (d) re-culturing the recovered spheres; (e) allowing the recovered spheres to associate with each other during said re-culturing; (f) dissociating the associated spheres to yield a suspension of single cells.

What provided is the above method, wherein Step (a) comprises culture on a low adherent container, or wherein Step (a) does not comprise culture on a low adherent container, or wherein Step (a) comprises culture on an ultra-low adherent container, or wherein Step (a) comprises culture on an ultra-low adherent container and not on a low adherent container.

Also, what is provided is above method, further comprising the step of acquiring a tumor sample prior to the step of dissociating the tumor sample to produce a cell suspension. Also, what is provided is above method, further comprising the step of establishing a proliferating adherent cell culture and expanding the cells.

The present invention provides an isolated population of cells originating from a human melanoma tumor, wherein at least 30% of the cells in the population express CD146 and wherein at least 30% of the cells in the population express CD146, or wherein at least 30% of the cells co-express CD146 and CD271, wherein the percent value is an average value over the population.

Also provided is the above population of cells, wherein at least 40% of the cells in the population express CD146 and at least 40% of the cells express CD271, or wherein at least 40% of the cells co-express CD146 and CD271, wherein the percent value is defined as an average value over the population.

Also provided is the above population of cells, wherein at least 50% of the cells in the population express CD146 and at least 50% of the cells express CD271, or wherein at least 50% of the cells co-express CD146 and CD271, wherein the percent value is an average value over the population.

Also provided are the above cells, wherein the culture on an adherent surface results in down-regulation of an immunosuppressive molecule in said population of expanded cells. Also provided are the above cells, wherein the culture on an adherent surface results in down-regulation of an immunosuppressive molecule, and (i) wherein the immunosuppressive molecule is at least one of indoleamine-pyrrole 2,3-dioxygenase, tumor growth factor-beta, and interleukin-10 (IL-10), and (ii) wherein the expression of the at least one immunosuppressive molecule prior to culture on adherent surface is 100%, and wherein down-regulation after culture on the adherent surface results in an expression that is at a level that is less than 80%, less than 70%, less than 60%, less than 50%, less than 40%, less than 30%, less than 20%, less than 10%, or less than about 80%, less than about 70%, less than about 60%, less than about 50%, less than about 40%, less than about 30%, less than about 20%, less than about 10%, and the like.

bFGF, or another growth factor, or bFGF in combination with one or more growth factors, can each be used at a concentration that is about 0.5 ng/mL, about 1.0 ng/mL, about 2.0 ng/mL, about 5.0 ng/mL, about 10 ng/mL, about 12 ng/mL, about 15 ng/mL, about 20 ng/mL, about 25 ng/mL, about 30 ng/mL, about 40 ng/mL, about 50 ng/mL, or in the range of 0.5-1.0 ng/mL, 1-2 ng/mL, 2-4 ng/mL, 1-5 ng/mL, 5-10 ng/mL, 10-12 ng/mL, 10-15 ng/mL, 15-20 ng/mL, 20-25 ng/mL, 25-30 ng/mL, 20-30 ng/mL, 30-40 ng/mL, and the like. What is also provided is exclusionary embodiments. For example, the present disclosure can exclude a method, and can exclude a medium, where bFGF occurs at 0.5 ng/mL, 1.0 ng/mL, 2.0 ng/mL, 5.0 ng/mL, 10 ng/mL, 12 ng/mL, 15 ng/mL, 20 ng/mL, 25 ng/mL, 30 ng/mL, 40 ng/mL, 50 ng/mL, or in the range of 0.5-1.0 ng/mL, 1-2 ng/mL, 2-4 ng/mL, 1-5 ng/mL, 5-10 ng/mL, 10-12 ng/mL, 10-15 ng/mL, 15-20 ng/mL, 20-25 ng/mL, 25-30 ng/mL, 20-30 ng/mL, 30-40 ng/mL, and the like. The above alternate embodiments, as well as the above exclusionary embodiments can be applied to a medium that is used with a non-adherent surface (or a very low-adherent surface, or an ultra-low adherent surface). Also, the above alternate embodiments, as well as the above exclusionary embodiments can be applied to a medium that is used with an adherent surface.

Furthermore, what is provided is the above cells, wherein none of the media used for culturing cells comprise an animal product. Also provided is the above cells, wherein the dispersing cells from one or both of the melanoma tumor sample, and from the microspheres, comprises treatment with an added protease. Also provided is the above cells, wherein the dispersing cells from the melanoma tumor sample, comprises added collagenase. What is also provided are the above cells, wherein the dispersing cells from the microspheres, comprises treatment with added trypsin.

The present disclosure provides an isolated population of cells, wherein at least 20%, at least 30%, at least 40%, at least 50%, at least 60%, at least 70%, at least 80%, at least 90%, or at least 95% of the cells express CD146, or wherein at least 20%, at least 30%, at least 40%, at least 50%, at least 60%, at least 70%, at least 80%, at least 90%, or at least 95% of the cells co-express CD271, or wherein at least 20%, at least 30%, at least 40%, at least 50%, at least 60%, at least 70%, at least 80%, at least 90%, or at least 95% of the cell population expresses each of CD146 and CD271, in at least the same percentage, or wherein at least 20%, at least 30%, at least 40%, at least 50%, at least 60%, at least 70%, at least 80%, at least 90%, or at least 95% of the cells co-express both CD146 and CD271.

The disclosure encompasses the above isolated population of cells, wherein the cells are comprised by a sphere of cells, wherein the cells occur in the form of a sphere of cells, wherein the cells are not comprised by a sphere of cells, wherein the cells are not part of a sphere of cells, wherein the cells are in suspension, or wherein the cells are in a monolayer.

In another aspect, the disclosure encompasses the above isolated population of cells, wherein at least 10%, at least 20%, at least 30%, at least 40%, at least 50%, at least 60%, at least 70%, at least 80%, at least 90%, at least 95%, or at least 98%, of the isolated population of cells are cancer stem cells. Moreover, the disclosure provides the isolated population that contains at least 1 cancer stem cell, at least 10 cancer stem cells, at least 100 cancer stem cells, at least 1,000 cancer stem cells, at least 2,000 cancer stem cells, at least 5,000 cancer stem cells, at least 10,000 cancer stem cells, at least 20,000 cancer stem cells, at least 50,000 cancer stem cells, at least 100,000 cancer stem cells, at least 1×10⁶ cancer stem cells, at least 10×10⁶ cancer stem cells, at least 100×10⁶ cancer stem cells, at least 1×10⁹ cancer stem cells, at least 10×10⁹ cancer stem cells, at least 100×10⁹ cancer stem cells, or at least 1×10¹² cancer stem cells.

What is contemplated by the present disclosure, is the above population of cells, that is capable of stimulating an effective immune response against a cell expressing MAGE antigen, wherein said isolated population is contacted to at least one dendritic cell, wherein said isolated population is processed in vivo by at least one dendritic cell, and wherein an effective immune response occurs in the subject in response to administration of the at least one dendritic cell to a subject.

What is further embraced by the present disclosure, is the above isolated population of cells, that is capable of stimulating an effective immune response against a cell that is a melanoma cancer cell, a lung cancer cell, a breast cancer cell, a colorectal cancer cell, or a hepatocellular cancer cell, wherein said isolated population is contacted to at least one dendritic cell, wherein said isolated population is processed in vivo by at least one dendritic cell, and wherein an effective immune response occurs in the subject as a consequence of administering the at least one dendritic cell, wherein the dendritic cell is administered to a subject having melanoma, lung cancer, breast cancer, colorectal cancer, or hepatocellular cancer, respectively.

In another aspect, the disclosure provides the above isolated population of cells, wherein the effective immune response comprises one or more of: (a) cytotoxic T cell response against a cell of the respective tumor, (b) increased response as measured by intracellular cytokine staining assays, ELISPOT assays, or tetramer assays; (c) increased population number of antigen-specific CD8⁺ T cells, (d) increased population number of antigen-specific CD4⁺ T cells, (e) reduction in tumor burden by RECIST criteria, and (f) increased survival of the subject.

Furthermore, the disclosure provides the above isolated population of cells of wherein substantially all of the population express MAGE antigen; wherein about 95% of the population express MAGE antigen; wherein about 90% of the population express MAGE antigen; wherein about 80% of the population express MAGE antigen; wherein about 70% of the population express MAGE antigen; wherein about 60% of the population express MAGE antigen; wherein about 50% of the population express MAGE antigen; wherein about 45% of the population express MAGE antigen; and, wherein more than about 25% of the population express MAGE antigen.

In another composition of matter exemplary implementation, the present disclosure encompasses an isolated population of cells, wherein at least 20%, at least 30%, at least 40%, at least 50%, at least 60%, at least 70%, at least 80%, at least 90%, or at least 95%, of the cells expresses MAGE; wherein at least 20%, at least 30%, at least 40%, at least 50%, at least 60%, at least 70%, at least 80%, at least 90%, or at least 95% of the cells express CD146; or wherein at least 20%, at least 30%, at least 40%, at least 50%, at least 60%, at least 70%, at least 80%, at least 90%, or at least 95% of the cells co-express CD271; or wherein at least 20%, at least 30%, at least 40%, at least 50%, at least 60%, at least 70%, at least 80%, at least 90%, or at least 95% of the cell population expressed each of CD146 and CD271, in at least the same percentage, or wherein at least 20%, at least 30%, at least 40%, at least 50%, at least 60%, at least 70%, at least 80%, at least 90%, or at least 95% of the cells co-express both CD146 and CD271.

### DEFINITIONS

“Administration” as it applies to a human, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell.

An “agonist,” as it relates to a ligand and receptor, comprises a molecule, combination of molecules, a complex, or a combination of reagents, that stimulates the receptor. For example, an agonist of granulocyte-macrophage colony stimulating factor (GM-CSF) can encompass GM-CSF, a mutein or derivative of GM-CSF, a peptide mimetic of GM-CSF, a small molecule that mimics the biological function of GM-CSF, or an antibody that stimulates GM-CSF receptor. An antagonist, as it relates to a ligand and receptor, comprises a molecule, combination of molecules, or a complex, that inhibits, counteracts, downregulates, and/or desensitizes the receptor. “Antagonist” encompasses any reagent that inhibits a constitutive activity of the receptor. A constitutive activity is one that is manifest in the absence of a ligand/receptor interaction. “Antagonist” also encompasses any reagent that inhibits or prevents a stimulated (or regulated) activity of a receptor. By way of example, an antagonist of GM-CSF receptor includes, without implying any limitation, an antibody that binds to the ligand (GM-CSF) and prevents it from binding to the receptor, or an antibody that binds to the receptor and prevents the ligand from binding to the receptor, or where the antibody locks the receptor in an inactive conformation.

Unless expressly stated otherwise, or dictated otherwise by the context, the term “expression” encompasses the following. Expression encompasses the biosynthesis of mRNA, polypeptide biosynthesis, polypeptide activation, e.g., by post-translational modification, or an activation of expression by changing the subcellular location or by recruitment to chromatin. In other words, “increased expression” encompasses increased biosynthesis, or increased activity that is caused by phosphorylation, or an increased activity that is caused by migration from the cytosol to the nucleus.

Antigen presenting cells (APCs) are cells of the immune system used for presenting antigen to T cells. APCs include dendritic cells, monocytes, macrophages, marginal zone Kupffer cells, microglia, Langerhans cells, T cells, and B cells (see, e.g., Rodriguez-Pinto and Moreno (2005) Eur. J. Immunol. 35:1097-1105). Dendritic cells occur in at least two lineages. The first lineage encompasses pre-DC1, myeloid DC1, and mature DC1. The second lineage encompasses CD34⁺⁺CD45RA− early progenitor multipotent cells, CD34⁺⁺CD45RA⁺ cells, CD34⁺⁺CD45RA⁺⁺ CD4⁺ IL-3Ralpha⁺⁺ pro-DC2 cells, CD4⁺CD11c⁻ plasmacytoid pre-DC2 cells, lymphoid human DC2 plasmacytoid-derived DC2s, and mature DC2s (see, e.g., Gilliet and Liu (2002) J. Exp. Med. 195:695-704; Bauer et al. (2001) J. Immunol. 166:5000-5007; Arpinati et al. (2000) Blood 95:2484-2490; Kadowaki et al. (2001) J. Exp. Med. 194:863-869; Liu (2002) Human Immunology 63:1067-1071; McKenna et al. (2005) J. Virol. 79:17-27; Rossi and Young (2005) J. Immunol. 175:1373-1381; Banchereau and Palucka (2005) Nat. Rev. Immunol. 5:296-306).

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. Biomarkers include blood counts, metabolite levels in serum, urine, or cerebrospinal fluid, tumor cell counts, cancer stem cell counts, tumor levels. Tumor size and number can be determined by the RECIST criteria (Eisenhauer et al. (2009) Eur. J. Cancer. 45:228-247). Expression markers encompass genetic expression of mRNA or gene amplification, expression of an antigen, and expression of a polypeptide. Clinical parameters include progression-free survival (PFS), 6-month PFS, disease-free survival (DFS), time to progression (TTP), time to distant metastasis (TDM), and overall survival, without implying any limitation.

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, epitope tags fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

The term, “originating,” as in, a population of cells that is, “originating from a human melanoma tumor,” encompasses, without implying any limitation, a population of cells that originated from a single cell from the tumor, and where the population of cells was produced by culturing the single cell to produce, by way of cell division, a population of cells. Also encompassed, is a population of cells originating from a number (number greater than one) of cells from one tumor, and where the number of cells was cultured to produce, by way of cell division, a greater number of cells. Also encompassed, is a population of cells that originated from one or more cells acquired from one particular tumor in a patient, and also from one or more cells acquired from a different tumor from the same patient, where the eventually produced population of cells represents the combined tumor cells from all of the harvested tumors. The number of tumors harvested can be one, two, three, four, or more. The term, a population of cells “originating from a human melanoma tumor” encompasses using as a starting cell, a melanoma tumor cell that happens not to be residing in a tumor, that is, starting with a melanoma tumor cell occurs as a solitary cell, e.g., one that resides in the lymphatic system or in the circulatory system. The term, “originating,” encompasses, without limitation, a harvested tumor cell that was subjected to purification by removing contaminating cells, subjected to culturing in a medium, subjected to storage in a refrigerator, subjected to expansion in a medium, subjected to in vitro formation of one or more spheres, and the like.

### Immunology of Cancer

Cancer is distinguished by the lack of effective immune response against the cancer. Lack of immune response can result, for example, from the fact that many tumor antigens are “self-antigens,” from lack of expression of MHC by the tumor cells and consequent lack of presentation of tumor antigens by the tumor cells, from the association of macrophages with tumors where the macrophages express cytokines that reduce immune response, and from the immunosuppressive activity of T regulatory cells (Tregs). Lack of immune response against tumors also results from the fact that tumor cells tend not to express molecules that stimulate innate immune response, that is, molecules that stimulate toll-like receptors (TLRs) or nucleotide-binding oligomerization domain (NOD)-like receptors). Cancer encompasses solid tumors as well as the hematological cancers, such as the leukemias and the myelodysplastic syndromes.

Cancer can be classified as a disorder of the immune system. This classification is based on the fact that the immune system fails, at least in certain segments of the afflicted human population, to respond optimally to cancer. Cancer cells avoid attack by the immune system because of the following reasons. First, cancer cells consist mainly of self-antigens, in striking contrast to the situation with infectious organisms. Some antigens that are classified as cancer antigens, are actually normal antigens that are overexpressed, or normal antigens that have a mutation in only one or two amino acids in the polypeptide chain. Second, cancer cells down-regulate Major Histocompatibility Complex (MHC), and thus do not much present tumor cell-derived peptides by way of MHC. Third, cancer cells, and associated tumor-associated macrophages, express cytokines that dampen immune response (see, e.g., Yu et al (2007) Nature Rev. Immunol. 7:41-51). This dampening is caused, for example, by the secretion of interleukin-10 (IL-10) by the cancer cells or by the associated macrophages. Fourth, unlike the situation with infections, cancer cells do not provide any immune adjuvant. Pathogens express a variety of naturally-occurring immune adjuvants, which take the form of toll-like receptor (TLR) agonists and NOD agonists (see, e.g., Kleinnijenhuis et al (2011) Clin. Dev. Immunol. 405310 (12 pages)). Generally, optimal activation of dendritic cells requires contact of an immune adjuvant with one or more toll-like receptors (TLRs). This refers to TLRs that are expressed by the dendritic cell. Hence, it is not likely the case that any cancer cell, or cancer cell antigen, without more, can optimally activate any dendritic cell. And without activation of the dendritic cell, contact between the dendritic cell and T cells (immune synapse) fails to result in optimal activation of the T cell.

In exemplary implementations, the present disclosure encompasses reagents and methods for activating dendritic cells (DCs), with one or more immune adjuvants, such as a toll-like receptor (TLR) agonist, e.g., CpG-oligonucleotide (TLR9), imiquimod (TLR7), poly(I:C) (TLR3), glucopyranosyl lipid A (TLR4), murein (TLR2), flagellin (TLR5), as well as an adjuvant such as CD40 agonists, e.g., CD40-ligand, or the cytokine, interferon-gamma, prostaglandin E2, and the like. See, e.g., U.S. Pat. No. 7,993,659 issued to Noelle et al; U.S. Pat. No. 7,993,648 issued to Kedl et al; U.S. Pat. No. 7,935,804 issued to Dubensky et al, each of which is incorporated herein by reference in its entirety. The present disclosure encompasses in vitro treatment of DCs with one or more of the above adjuvant reagents, or in addition, or alternatively, administration of the adjuvant to a human subject, animal subject, or veterinary subject.

The immune system encompasses cellular immunity, humoral immunity, and complement response. Cellular immunity includes a network of cells and events involving dendritic cells, CD8⁺ T cells (cytotoxic T cells; cytotoxic lymphocytes), and CD4⁺ T cells (helper T cells). Dendritic cells (DCs) acquire polypeptide antigens, where these antigens can be acquired from outside of the DC, or biosynthesized inside of the DC by an infecting organism. The DC processes the polypeptide, resulting in peptides of about ten amino acids in length, transfers the peptides to either MHC class I or MHC class II to form a complex, and shuttles the complex to the surface of the DC. When a DC bearing a MHC class I/peptide complex contacts a CD8⁺ T cell, the result is activation and proliferation of the CD8⁺ T cell. Regarding the role of MHC class II, when a DC bearing a MHC class II/peptide complex contacts a CD4⁺ T cell, the outcome is activation and proliferation of the CD4⁺ T cell (Munz et al. (2010) Curr. Opin. Immunol. 22:89-93; Monaco (1995) J. Leukocyte Biol. 57:543-547; Robinson et al (2002) Immunology 105:252-262). Although dendritic cells presenting antigen to a T cell can “activate” that T cell, the activated T cell might not be capable of mounting an effective immune response. Effective immune response by the CD8⁺ T cell often requires prior stimulation of the DC by one or more of a number of interactions. These interactions include direct contact of a CD4⁺ T cell to the DC (by way of contact the CD4⁺ T cell's CD40 ligand to the DC's CD40 receptor), or direct contact of a TLR agonist to one of the dendritic cell's toll-like receptors (TLRs).

Humoral immunity refers to B cells and antibodies. B cells become transformed to plasma cells, and the plasma cells express and secrete antibodies. Naïve B cells are distinguished in that they do not express the marker CD27, while antigen-specific B cells do express CD27 (Perez-Andres et al. (2010) Cytometry Part B 78B (Suppl. 1) S47-S60). The secreted antibodies can subsequently bind to tumor antigens residing on the surface of tumor cells. The result is that the infected cells or tumor cells become tagged with the antibody. With binding of the antibody to the infected cell or tumor cell, the bound antibody mediates killing of the infected cell or tumor cell, where killing is by NK cells. Although NK cells are not configured to recognize specific target antigens, in the way that T cells are configured to recognize target antigens, the ability of NK cells to bind to the constant region of antibodies, enables NK cells to specifically kill the cells that are tagged with antibodies. The NK cell's recognition of the antibodies is mediated by Fc receptor (of the NK cell) binding to the Fc portion of the antibody. This type of killing is called, antibody-dependent cell cytotoxicity (ADCC). NK cells can also kill cells independent of the mechanism of ADCC, where this killing requires expression of MHC class I to be lost or deficient in the target cell (see, e.g., Caligiuri (2008) Blood 112:461-469).

The present disclosure, in some exemplary implementations, provides reagents and methods to enhance NK cell-mediated killing of cancer stem cells. NK cells can mediate cytotoxicity against cancer stem cells (see, e.g., Jewett and Tseng (2011) J. Cancer. 2:443-457). Without wishing to be bound to any particular mechanism, the disclosure encompasses administration of cancer stem cell antigens, or administering dendritic cells loaded with cancer stem cell antigens, where the antigens stimulate the production of antibodies that specifically recognize one or more of the cancer stem cell antigens, and where the antibodies mediate ADCC. The phrase, loaded with antigens, refers to the ability of the dendritic cell to capture live cells, to capture necrotic cells, to capture dead cells, to capture polypeptides, or to capture peptides, and the like. Capture by cross-presentation is encompassed by the present disclosure. Also encompassed, is the use of antigen-presenting cells that are not dendritic cells, such as macrophages or B cells (see, e.g., O'Neill et al (2004) Blood. 104:2235-2246; Sabado and Bhardwaj (2010) Immunotherapy. 2:37-56).

The technique of “delayed type hypersensitivity response” can be used to distinguish between immune responses that mainly involve cellular immunity or mainly involve humoral immunity. A positive signal from the delayed type hypersensitivity response indicates a cellular response (see, e.g., Roychowdhury et al. (2005) AAPS J. E834-E846).

The disclosure encompasses differential trypsinization, for example, treatment using 0.25% trypsin for ten minutes. Also encompassed, is complete trypsinization, for example, incubating with 0.25% trypsin or 120 minutes (Liu et al (2012) PLoS ONE. 7:e35720 (14 pages). In another aspect, the disclosure excludes reagents or methods that use added trypsin, that use differential trypsinization, or that use complete trypsinization. The disclosure encompasses reagents or methods, and also excludes one or more of the reagents or methods, as described in US2012/0122215 of Edinger et al; 2012/0020936 of Harira; 2011/0250182 of Abbot et al, which are each incorporated herein by reference in their entirety. Selvan et al (2010) Melanoma Res. 20:280-292, disclose reagents and methods for detaching adherent cells.

The disclosure provides pharmaceuticals, reagents, kits including diagnostic kits, that wherein the pharmaceuticals, reagents, and kits, comprise dendritic cells, antibodies, or antigens. What is also provided are methods for administering compositions that comprise at least one dendritic cell and at least one antigen, methods for stimulating antibody formation, methods for stimulating ADCC, methods for stimulating complement-dependent cytotoxicity, and methods and kits for determining patient suitability, for determining patient inclusion/exclusion criteria in the context of a clinical trial or ordinary medical treatment, and for predicting response to the pharmaceutical or reagent. Complement-dependent cytotoxicity is described (see, e.g., Goodman et al. (1990) J. Clin. Oncol. 8:1083-1092; Cheson (2010) J. Clin. Oncol. 28:3525-3530). The pharmaceutical compositions, reagents, and related methods, of the disclosure encompass CD83 positive dendritic cells, where CD83 is induced by loading with IFN-gamma-treated cancer cells. In a CD83 aspect of the disclosure, the CD83 is induced by at least 2%, at least 3%, at least 4%, 6%, 7%, 8%, 9%, 10%, and the like. In another aspect, what is excluded are DC reagents, or DC-related methods, where CD83 of dendritic cells is not detectably induced by loading with IFN-gamma-treated cancer cells. Media, labeled antibodies, cell culturing supplies, and other reagents are available from, e.g., Sigma-Aldrich, St. Louis, Mo., Life Technologies, Carlsbad, Calif., and GIBCO, Grand Island, N.Y. KO DMEM medium is “Knockout Dulbecco's modified Eagle's medium.” B27 medium is described, e.g., in Stevens et al (2009) Proc. Nat'l. Acad. Sci. 106:16568-16573, and Brewer et al (1993) J. Neurosci. Res. 35:567-576. Glutamax® is L-alanyl-L-glutamine.

### Loading Dendritic Cells

Dendritic cells (DCs) can be loaded with melanoma tumor cell antigen, DC vaccines can be prepared, and DC vaccines can be administered to a human subject by one or more routes of administration. See, e.g., Selvan et al (2008) Int. J. Cancer. 122:1374-1383; Sabado and Bhardwaj (2010) Immunotherapy. 2:37-56; Hirschowitz et al (2004) J. Clin. Oncol. 22:2808-2815; O'Neill et al (2004) Blood. 104:2235-2246; Schwaab et al (2009) Clin. Cancer Res. 15:4986-4992; Zhong et al (2007) Clin. Cancer Res. 13:5455-5462.

The present disclosure provides compositions and methods, where tumor cells are inactivated, e.g., by radiation, nucleic acid cross-linkers, polypeptide linkers, or combinations of these. One particular nucleic acid alkylator is beta-alanine, N-(acridin-9-yl), 2-[bis(2-chloroethyl)amino]ethyl ester. Exemplary cross-linkers, such as psoralens in combination with ultraviolet (UVA) irradiation, have the ability to cross-link DNA but to leave proteins unmodified. Nucleic acid targeting compound can be 4′-(4-amino-2-oxa)butyl-4,5′,8-trimethylpsoralen (“S-59”). Cells can be inactivated with 150 micromolar of psoralen S-59 and 3 J/cm² UVA light (FX 1019 irradiation device, Baxter Fenwal, Round Lake, Ill.). See, U.S. Pat. No. 7,833,775 of Dubensky and U.S. Pat. No. 7,691,393 of Dubensky, which are incorporated herein by reference, in their entirety.

### Tumor Antigens

The present disclosure provides reagents and methods for stimulating immune response against a tumor antigen, for stimulating immune response against a cell expressing a tumor antigen, for administering to a human or veterinary subject, and for use in diagnosing a human or veterinary subject, and the like. The present disclosure provides a reagent, and related methods, for stimulating immune response against a cell that expresses one or more of, e.g., p53, MUC1, NY-ESO-1, c-myc, surviving, p62, cyclin B1, and Her2/neu (see, e.g., Reuschenbach et at (2009) Cancer Immunol. Immunother. 58:1535-1554). In some exemplary implementations, the immune response is against a cell that expresses said antigen or antigens, but not necessarily specific to that cell (the immune response is against other cells as well). In in other exemplary implementations, the immune response is against a cell that expresses said antigen or antigens, and where the immune response requires recognition of said antigen. In yet other exemplary implementations, the immune response is against a cell that expresses said antigen or antigens, and where the immune response does not require recognition of said antigen.

The present disclosure provides reagents and methods for stimulating immune response against a cell that expresses heat shock protein (HSP). Immunotherapy against HSP is effective against colorectal cancer, melanoma, and renal cell carcinoma (see, e.g., Buonaguro et al (2011) Clinical Vaccine Immunol. 18:23-34). What is encompassed is reagent and method for stimulating immune response against cancer-testis antigen, or against differentiation antigen, or against an overexpressed antigen, or against tumor-associated carbohydrate antigen. In other exemplary implementations, reagent and method that stimulates immune response against a neoplastic disorder that expresses MUC1, an antigen that is associated with breast, colorectal, gastric, pancreatic, and ovarian cancer (Reuschenbach et al (2009) Cancer Immunol. Immunother. 58:1535-1554). Also provided is reagent and method that stimulates immune response against a neoplastic disorder that expresses p53, an antigen associated with lung cancer, colorectal cancer, esophageal cancer, and ovarian cancer (Reuschenbach et al, supra). Moreover, what is provided is reagent and method that stimulates immune response against a neoplastic disorder that expresses Her2/neu, an antigen associated with breast, colorectal, and ovarian cancer. In exemplary implementations, the present disclosure provides reagents and methods for stimulating immune response against the following antigen, or against a cell expressing said antigen, where the antigen is a MAGE family antigen. MAGE means, “melanoma associated antigen.” MAGE family antigens are associated with melanoma (Selvan et al (2008) Int. J. Cancer. 122:1374-1383), as well as with hepatocellular carcinoma (see, e.g., Mou et al (2002) Brit. J. Cancer. 86:110-116), ovarian cancer (Zhang et al (2010) BMC Cancer. 10:163 (6-pages), non-small cell lung cancer (NSCLC) (Gridelli et al (2009) The Oncologist. 14:909-920; Sienel et al (2007) Clin. Cancer Res. 13:3840-3847), and colorectal cancer (Toh et al (2009) Clin. Cancer Res. 15:7726-7736).

CD133 is an antigen expressed by a variety of cancers, including melanoma, colorectal cancer, Ewing's sarcoma, hepatocellular cancer (HCC), non-small cell lung cancer (NSCLC), and ovarian cancer (Perego et al (2011) J. Inv. Dermatol. 11:546-547; Cao, et al. (2011) BMC Gastroenterol. 11:71 (11 pages); Lorico and Rappa (2011) 135039 (6 pages); Ferrandina et al (2009) BMC Cancer. 9:221 (9 pages)). The present disclosure provides a population of cancer stem cells that expresses CD133; at least one dendritic cell loaded with a cancer stem cell that expresses CD133; methods for preparing a dendritic cell loaded with a cancer stem cell that expresses CD133; and methods of administering at least one dendritic cell loaded with a cancer stem cell that expresses CD133 to a subject that has a cancer that expresses the CD133 biomarker.

Regarding the ABCB5 antigen, the present disclosure provides reagents and methods, where cancer stem cells expressing ABCB5 are contacted to, or loaded onto, dendritic cells, and where the loaded dendritic cells are administered to a human subject or animal that has an ABCB5-expressing cancer. ABCB5 expression is associated, for example, with melanoma cancer stem cells (Schatton et al (2010) Cancer Res. 70:697-708), as well as with colorectal cancer (Wilson et al (2011) Cancer Res. 71:5307-5316). Related methods include inducing immune response against a cell that expresses ABCB5, preferably, a cancer stem cell that expresses ABCB5.

The present disclosure also encompasses reagents and methods, relating to the following antigens: aldehyde dehydrogenase (ALDH), for example ALDH1A3; ABCB1 (P-glycoprotein/MDR1); BCL2A1; SNAI2 (slug); ATM, CHEK1, and CHEK2. Also encompassed are reagents and methods, relating to CD44, CD133, CD24, CD49f, ESA; CD166; and lineage panels. Lineage panels include CD45, CD31, CD3, CD64, CD10, CD16, CD18, and GPA; CD45, CD31, CD140a, and Ter119; CD45, CD31 and CD140a. Typically lineage panels include one or more of CD45, CD31, CD3, CD64, CD10, CD16, CD18, GPA, CD140a and Ter119 (US 2011/0124032 of Diehn et al, which is hereby incorporated by reference in its entirety).

What is also encompassed is reagent, and related methods, that are specific for stimulating immune response against one or more of the following antigens, or against a cell expressing one or more of the following antigens: MAGE-A subtypes, such as, MAGE-A1, MAGE-A2, MAGE-A3/6, MAGE-A4, and MAGE-A12 (see, e.g., Sienel et al, supra). In alternative exemplary implementations, the disclosure provides reagents and related methods that stimulate against intercellular adhesion molecule-1 (ICAM-1), or against a cell expressing ICAM-1, or against a neoplastic cell, or against a cancer in a subject, that is identified with one or more of ICAM-1. ICAM-1 associated cancers include melanoma, colon cancer, bladder cancer, lung cancer, pancreatic cancer, and hepatocellular carcinoma (Shih et al (2004) Korean J. Intern. Med. 19:48-52). Moreover, the present disclosure provides reagents and methods for stimulating immune response against the following antigen, or against a cell expressing the following antigen, or against a neoplastic cell, or against a cancer in a subject that is identified with sHLA-E. sHLA-E is a non-classical MHC class I molecule, that is associated with melanoma, colorectal cancer, and renal cancer (Allard et al (2011) PLoS One. 6:e21118 (9-pages). Also provide are reagents and method for stimulating response against the following antigen, or against a cell that expresses the following antigen, or against a neoplastic cell expressing the following antigen, or against a cancer in a subject that expresses the following antigen. The antigen is HERV-K gag-related NGO-Pr-54. This antigen is associated with ovarian cancer, prostate cancer, and leukemia (Ishida et al (2008) Cancer Immunol. 8:15 (10-pages)).

The present disclosure provides reagents and related methods for stimulating immune response against a neoplastic cell that is as follows, or against a cancer in a subject that is as follows. The exemplary implementations encompass, and are not limited to: (1) melanoma and colorectal; (2) melanoma and ovarian; (3) melanoma and lung; (4) melanoma and hepatic; (5) melanoma, colorectal, and ovarian; (6) melanoma, colorectal, and lung; (7) melanoma, colorectal, and hepatic; (8) melanoma, lung, and hepatic; (9) melanoma, ovarian, and lung; (10) melanoma, ovarian, and hepatic; (11) melanoma, ovarian, lung, and hepatic; (12) melanoma, colorectal, lung, and hepatic; (13) melanoma, colorectal, ovarian, and hepatic; (14) melanoma, colorectal, ovarian, and lung; and (15) melanoma, colorectal, ovarian, lung, and hepatic.

Exclusionary exemplary implementations are reagents and methods that do not induce, or have been shown to fail to induce, a pre-determined level of immune response. The pre-determined level of immune response can be assessed, for example, against one or more of a cancer cell that is melanoma cell, colorectal cancer cell, ovarian cancer cell, lung cancer cell, or hepatic cancer cell. By way of a non-limiting definition, the pre-determined level stimulation can be, for example, a stimulation that is less than 20%, less than 15%, less than 10%, less than 5%, less than 2%, less than 1%, a maximal level. The maximal level can be in terms of percent of human subjects showing maximal response by RECIST criteria, in terms of killing of cancer stem cells in a human subject or experimental animal, in terms of overall survival, in terms of progression-free survival (PFS); in terms of time to progression (TTP), in terms of a maximal cytotoxic lymphocyte (CTL) response signal, in terms of a maximal ELISPOT assay signal, in terms of a maximal result from antibody dependent cell cytotoxicity (ADCC), in terms of T cell activation, in terms of T cell expansion, in terms of intracellular cytokine staining (ICS) assays, in terms of tetramer assays, and the like (see, e.g., Nomura et al (2008) Cytometry A. 73:984-991). For example, in one exemplary implementation, what is excluded are reagents and methods that stimulate less than 20% of a pre-determined maximal level. Clinical endpoints, such as PFS, TTP, time to distant metastasis, overall survival, and techniques for interpreting these endpoints, are detailed (Brody (2012) Clinical Trials: Study Design, Endpoints and Biomarkers. Elsevier, San Diego, Calif.), and are part of the present disclosure.

Reagents, methods, and techniques that are encompassed by the present disclosure include, US 2011/0313229 of Sugaya et al, which concerns cancer stem cells, WO 2011/041453 of Weismann and Boiko, which concerns isolation of melanoma cancer stem cells, and US 2011/0286963 of Blot-Chabaud et al, which concerns CD146. Each of these is hereby incorporated by reference in their entirety.

Non-adherent conditions, non-adherent plates, non-adherent coatings, and the like, can be provided by hydrophobic materials and by non-biofouling materials, such as polystyrenes, thin agar coating, siloxanes, fluorpolymers, polyethylenes, and the like. See, e.g., Tsai et al (2009) J. Biomater. Sci. Polym. Ed. 20:1611-1628; U.S. Pat. No. 7,790,217 issued to Toreki et al, U.S. Pat. No. 6,342,591 issued to Zamora et al, US 2011/0282005 of Jiang et al, each of which is incorporated herein in its entirety. Polyethyleneglycol (PEG) for non-adhesion is disclosed by Kim et al (2006) Lab Chip. 6:1432-1437. Ultra-Low Attachment surfaces include Corning Ultra-Low Attachment Surface (Corning, Inc.) and Thermo Scientific's Nunc HydroCell Surface.

In exemplary implementations, the disclosure provides additives that can promote non-adherent conditions, such as additives that are membrane expanders, tensioactive agents, Pluronic F-68, Tween-80, or polyvinylalcohol (PVA) (Sigma Aldrich catalogue, St. Louis, Mo.).

In exemplary implementations, down-regulation of indoleamine-pyrrole 2,3-dioxygenase can be effected by sodium butyrate, COX-2 inhibitors, anti-sense nucleic acids, si-RNA, or micro-RNA. Down-regulation of IL-10 or TGF-beta can be affected by anti-sense nucleic acids, si-RNA, or micro-RNA. Liu et al (2011) FEBS Lett. 585:1963-1968, discloses the use of micro-RNA to down-regulated IL-10 expression. Yu et al (2012) Carcinogenesis. 33:68-76, disclose the use of micro-RNA to decrease efficacy of transforming growth factor-beta (TGF-beta), by down-regulating TGF-beta receptor. Lang et al (2011) Biochim. Biophys. Res. Commun. 409:448-453, report the use of small interference RNA (siRNA) to inhibit TGF-beta expression.

In exemplary implementations, expansion procedure is conducted starting with a single cell. In other exemplary implementations, expansion procedure is initiated with about 10 cells, about 20 cells, about 50 cells, about 100 cells, about 200 cells, about 500 cells, about 1000 cells, about 2000 cells, about 5000 cells, about 10000 cells, about 20000 cells, about 50000 cells, and the like.

Exclusionary exemplary implementations are provided. Without implying any limitation, the reagents and method of the present disclosure can exclude a population of cells, a tissue, an organ, or a subject, and the like, where expression of CD146 is less than 50%, less than 40%, less than 30%, less than 20%, less than 15%, less than 10%, less than 5%, or less than 2%. Also, what can be excluded is a population of cells, a tissue, an organ, or a subject, and the like, where expression of CD271 is less than 50%, less than 40%, less than 30%, less than 20%, less than 15%, less than 10%, less than 5%, or less than 2%. In yet another exclusionary exemplary implementation, what can be excluded is a population of cells, a tissue, an organ, or a subject, and the like, where co-expression of both CD146 and CD271 (co-expression in exactly the same cell, for each cell measured) is less than 50%, less than 40%, less than 30%, less than 20%, less than 15%, less than 10%, less than 5%, or less than 2%. Also, what can be excluded is a population of cells, a tissue, an organ, or a subject, and the like, where expression of both CD146 and CD271 (either co-expressed in exactly the same cell, or merely both expressed in the entire population of cells) is less than 50%, less than 40%, less than 30%, less than 20%, less than 15%, less than 10%, less than 5%, or less than 2%.

The term “co-express” refers to the situation where the indicated markers, e.g., genes, polypeptides, antigens, and so on, are expressed in exactly the same cell, and also are expressed concurrently. Regarding concurrent co-expression, the time frame of co-expression can be about 5 minutes, about 30 minutes, about 1 hour, about 6 hours, about 12 hours, about 1 day, about 2 days, about 4 days, about 8 days, and so on. The time frame of co-expression can be at least 1 minute, at least 5 min, at least 10 min, at least 20 min, at least 60 min, at least 2 hours, at least 4 hours, at least 6 hours, at least 12 hours, at least 24 hours, at least 2 days, at least 3 days, at least 4 days, at least 8 days, at least one week, at least two weeks, and so on.

The present disclosure isolated population of cells originating from a human melanoma tumor, wherein at least 20% of the cells in the population express CD146 and at least 20% of the cells express CD271, or wherein at least 20% of the cells co-express CD146 and CD271. Also provided, is isolated population of cells originating from a human melanoma tumor, wherein at least 30% of the cells in the population express CD146 and at least 30% of the cells express CD271, or wherein at least 40% of the cells co-express CD146 and CD271. Also provided, is isolated population of cells originating from a human melanoma tumor, wherein at least 40% of the cells in the population express CD146 and at least 30% of the cells express CD271, or wherein at least 40% of the cells co-express CD146 and CD271. The present disclosure encompasses, without limitation, a population of cells that occurs as a monolayer or other layer, a population of cells that occurs as a suspension, a population of cells that occurs as one or more spheres, and so on.

### Populations of Cells Detectably Expressing Only One of CD146 or CD271

The present disclosure encompasses a population of cells that expresses CD146 but not CD271. Also, the present disclosure encompasses a population of cells that expresses CD271 but not CD146. In embodiments, what is encompassed is a population of cells where at least 20% of the cells express CD146, where at least 30%, at least 40%, at least 50%, at least 60%, at least 70%, at least 80%, at least 90%, at least 95%, at least 98%, of the cells express CD146 but not CD271. Also, what is encompassed is a population of cells where at least 20% of the cells express CD146, where at least 30%, at least 40%, at least 50%, at least 60%, at least 70%, at least 80%, at least 90%, at least 95%, at least 98%, of the cells express CD1271 but not CD146. What is encompassed are methods of culturing the above cells, methods of isolating the cells, methods of loading the cells on dendritic cells, vaccines comprising the above cells, vaccines comprising dendritic cells loaded with the above cells, methods for administering the vaccines to subject, and so on.

Melanoma cells that are CD146+/CD271− identify mesenchymal cancer cells, and melanoma cells that are CD146+/CD271+ identify cells that are cancer stem cells with mesenchymal characteristics. The present disclosure provides a population of cells, and related methods, wherein at least 20% of the cells in the population are CD146+/CD271−, at least 22%, at least 24%, at least 26%, at least 28%, at least 30%, at least 34%, at least 38%, at least 42%, at least 46%, at least 50%, at least 54%, at least 58%, at least 62%, at least 66%, at least 70%, at least 74%, at least 78%, or at least 82%, of the cells are CD146+/CD271−. The present disclosure provides a population of cells, and related methods, wherein at least 20% of the cells in the population are CD146−/CD271+, at least 22%, at least 24%, at least 26%, at least 28%, at least 30%, at least 34%, at least 38%, at least 42%, at least 46%, at least 50%, at least 54%, at least 58%, at least 62%, at least 66%, at least 70%, at least 74%, at least 78%, or at least 82%, of the cells are CD146−/CD271+. In exclusionary embodiments, the present disclosure can exclude any cell population that fails to meet one of the above-disclosed percentages.

### Exclusionary Embodiments

What can be excluded is a single cell, a population of cells, a population of cells that occurs as a monolayer or other layer, a population of cells that occurs as a suspension, a population of cells that occurs as one or more spheres, and so on, where expression of CD146 occurs in less than 10% of the cells, occurs in less than 20%, less than 30%, less than 40%, less than 50%, less than 60%, less than 70%, less than 80%, of the cells. What can be excluded is a single cell, a population of cells, a population of cells that occurs as a monolayer or other layer, a population of cells that occurs as a suspension, a population of cells that occurs as one or more spheres, and so on, where expression of CD271 occurs in less than 10% of the cells, occurs in less than 20%, less than 30%, less than 40%, less than 50%, less than 60%, less than 70%, less than 80%, of the cells. What can be excluded is a single cell, a population of cells, a population of cells that occurs as a monolayer or other layer, a population of cells that occurs as a suspension, a population of cells that occurs as one or more spheres, and so on, where expression of each of CD146 and CD271 occurs in less than 10% of the cells, occurs in less than 20%, less than 30%, less than 40%, less than 50%, less than 60%, less than 70%, less than 80%, of the cells. What can be excluded is a single cell, a population of cells, a population of cells that occurs as a monolayer or other layer, a population of cells that occurs as a suspension, a population of cells that occurs as one or more spheres, and so on, where co-expression of each of CD146 and CD271 occurs in less than 10% of the cells, occurs in less than 20%, less than 30%, less than 40%, less than 50%, less than 60%, less than 70%, less than 80%, of the cells. In this context, co-expression means that, with analysis of a given particular cell, CD146 and CD271 are both detectably expressed by that particular cell. What can be excluded is any population of melanoma cells, where over 1%, over 2%, over 4%, over 5%, over 10%, over 15%, over 20%, over 30%, over 40%, over 50%, over 60%, over 70%, over 80%, over 90%, of the melanoma cells are not melanoma cancer stem cells.

### BRIEF DESCRIPTIONS OF THE FIGURES

FIG. 1. Flow cytometry characterization of melanoma stem cells at various stages of the purification process.

FIG. 2. Table showing percentages of expression of CD146 and CD271 in autologous melanoma cell lines.

FIG. 3. Phenotype (CD146; CD271) of various melanoma cell lines.

FIG. 4. Flow cytometry results (CD146; CD271) of melanoma stem cells in enzyme digest, melanoma stem cells prepared by standard method, and melanoma cells prepared by sphere-generating method.

FIG. 5. Flow cytometry results (CD146; CD271) (MHC Class II; MHC Class I) of melanoma cells subjected to various treatments.

FIG. 6. Drawing of purification procedure using Method 1.

FIG. 7. Drawing of purification procedure using Method 2.

FIG. 8. Enrichment of cancer stem cells during purification process. FIG. 8A shows histogram. FIG. 8B shows flow cytometry results.

FIG. 9. Histogram comparing expression of: (1) CD146 and CD271 in bulk tumor cells, (2) “Cancer stem cells” of the present disclosure, and (3) “Purified cell line” produced by standard method.

As used herein, including the appended claims, the singular forms of words such as “a,” “an,” and “the” include their corresponding plural references unless the context clearly dictates otherwise. All references cited herein are incorporated by reference to the same extent as if each individual publication, patent, published patent application, and sequence listing, as well as figures and drawings in said publications and patent documents, was specifically and individually indicated to be incorporated by reference.

### FURTHER DESCRIPTION

### Staging of Cutaneous Melanoma

The pharmaceutical or reagent of the disclosure can be administered to melanoma patients, where melanoma is diagnosed at Stage I, Stage II, Stage III, or Stage IV (Mohr et al (2009) Ann. Oncology (Suppl. 6) vi14-vi21). Stage I, for example, refers to patients with primary melanomas without evidence of regional or distant metastasis. Stage II includes patients without evidence of lymphatic disease or distant metastases, where the patients are further characterized, e.g., by lesions greater than 1 mm and less than or equal to 2 mm thick with ulceration of the overlying epithelium, or by lesions greater than 2 mm and less than or equal to 4 mm thick with epithelial ulceration. Stage III melanoma includes lesions with pathologically documented involvement of regional lymph nodes or in-transit or satellite metastases, where patients may have, e.g., one, two, three, or four or more affected lymph nodes. Stage IV melanoma is defined by the presence of distant metastases, where the metastasis is located only in distant skin, subcutaneous tissues, or lymph nodes, where the metastasis involves lung metastases, or where the metastasis involves all other visceral sites.

The disclosure encompasses methods for administration that are preventative, that is, for use with subjects not yet or never diagnosed with a melanoma. What is encompassed are methods for administration where a subject had earlier been diagnosed with a melanoma, and had earlier been successfully treated to eradicate the melanoma (or had experienced a spontaneous complete remission), and where following eradication the administration is used preventatively.

The disclosure provides a pharmaceutical composition or pharmaceutical reagent, related methods of administration, and methods of treatment, that result in survival data with a hazard ratio (HR) of less than 1.0, HR less than 0.9, HR less than 0.8, HR less than 0.7, HR less than 0.6, HR less than 0.5, HR less than 0.4, HR less than 0.3, and the like. The disclosure results in overall survival data, progression-free survival data, time to progression data, and so on. What is also provided is 6-month PFS of at least 40%, at least 50%, at least 60%, at least 70%, at least 75%, at least 80%, at least 85%, at least 90%, at least 95%, and so on. Moreover, what is provided is 6-month overall survival of at least 40%, at least 50%, at least 60%, at least 70%, at least 75%, at least 80%, at least 85%, at least 90%, at least 95%, and so on. Additionally, what is provided is 1-year (or 2-year) PFS of at least 40%, at least 50%, at least 60%, at least 70%, at least 75%, at least 80%, at least 85%, at least 90%, at least 95%, and so on. Moreover, what is provided is 1-year (or 2-year) overall survival of at least 40%, at least 50%, at least 60%, at least 70%, at least 75%, at least 80%, at least 85%, at least 90%, at least 95%, and so on (see, e.g., U.S. Dept. of Health and Human Services. Food and Drug Administration. Guidance for Industry. Clinical trial endpoints for the approval of cancer drugs and biologics (April 2005)).

### Biomarkers and Flow Cytometry

Studies of melanoma cancer stem cells disclose reagents and methods for detecting markers, such as CD271 (Civenni et al (2011) Cancer Res. 71:3098-3109), CD146, CD146 (Perego et al (2010) J. Inv. Dermatol. 130:1877-1886); and ABCB5 (Schatton et al (2011) Cancer Res. 70:697-708). Other biomarkers of interest include CD20, CD133, CD44, CD90, CD24, EpCAM, ALDH1, and ABCB5 (see, e.g., Wang and Jacob (2011) Genome Medicine. 3:11 (6 pages); Schlaak et al (2012) Oncotarget. 3:22-30).

Phenotypic characterization of the cell populations are performed using monoclonal antibodies against surface markers (BD Pharmingen San Diego, Calif.: BD) Pharmingen. CaliBRITE flow cytometry calibration (BD Pharmingen) is used prior to each run and the same instrument settings were used throughout the collection of flow cytometric data. Flow cytometry is conducted with a Beckton-Dickenson FACS Calibur® flow cytometer. The number of polypeptides expressed by a cell can be measured, for example, using fluorescent antibodies with quantitation by flow cytometry (see, e.g., Macey (2010) Flow Cytometry: Principles and Applications, Humana Press; Hawley (2010) Flow Cytometry Protocols (Methods in Molecular Biology) Humana Press; Shapiro (2003) Practical Flow Cytometry, Wiley-Liss).

Characterization of cell lines of the present disclosure by flow cytometry demonstrated the enrichment for cells of mesenchymal and neural crest origin (CD146 and CD271, respectively) which have been described as melanoma stem cell markers. Comparison of these cell lines versus the original bulk enzyme digest samples demonstrated that they were enriched for either CD146 and/or CD271 (78.5±8.3% versus 26.9±5.8%) after purification and expansion. Examination of 35/42 cell lines used in a randomized phase II clinical trial revealed consistent expression these markers in the purified tumor cell lines (35.2±3.9% CD146+/CD271−, 41.5±4.3 CD146+/CD271+, 16.9±4.0 CD146−/CD271−, 6.4±1.9 CD146−/CD271+). Using these cells as the antigen source in an autologous dendritic cell therapy resulted in 50% 5-year survival in patients with stage IV melanoma (n=54). Using excess cryopreserved samples with our methods, we were able to reduce the production time to 2 months and increase the success rate to 80%. This process also resulted in increasing the purity of the cancer stem cells from ˜70% to >90% based on these known cancer stem cell markers. In addition, contaminating fibroblasts were eliminated with minimal skilled manipulations. This approach is suitable for automation and/or optimization where a closed and uniform systems may be constructed that support automation and scalability. Scalability and optimization is often associated with reductions in cost of delivery and preparation, automation may also result in reduced cost of labor.

### Spheres

The ability of cells to form spheres result, in part, from cell-surface proteins called, integrins.” Homophilic integrins expressed on the cell's surface ensure that cells “stay together”. Spheres are formed directly from enzyme digest (ED) which is a single cell suspension at the very beginning of a culture, or can be formed from frozen sample or an existing attached culture at any time. The enzyme digest seeding result in this spherical formations that incorporate the cells with the specific surface properties. Fibroblasts for example cannot be incorporated, eventually are faded out from a culture during gravitational feeding. The media used is lacking of molecules that promote adhesion in order to prevent the non-specific agglomeration of the cells not having homophilic proprieties and to prevent the adhesion to the culture vessel surfaces. Such adhesion molecules (CAMs) are commonly found in the animal or human serum, therefore a media composition which is serum free is suitable.

In the serum free media culture, what is supplied by way of supplements to the media include any hormones, nutrients, mineral, and vitamins that are required for supporting growth and maintenance, or other desired aspects of cell physiology and function. In some instance one can stimulate and sustain the stem cell proliferation with the addition or adjustment of amount of growth factors that possess a mitogenic activity, e.g., such as FGF family and EGF.

Spheres of cells, including spheres of cancer stem cells, can be characterized in terms of biomarker expression by way of fixing and staining with labeled antibodies, followed by viewing with confocal microscopy (Weiswald et al (2010) Cancer. 10:106 (11 pages)). Spheres can be prepared, for example, from suspensions obtained from fresh tumors, or from cells adapted to grow as adherent cells, as documented for the case of melanoma cells (Perego et al (2011) J. Inv. Dermatol. 11:546-547). Spheres can be generated from a single cell, as shown by the fluorescent microscopy images of spheres (see, e.g., Cao et al (2011) BMC Gastroenterol. 11:71 (11 pages)). The morphology of spheres, for example, large and irregular versus tiny and compact, may be influenced by the choice of medium (Mancini et al (2011) PLoS ONE. 6:e21320 (12 pages). Without implying any limitation, the present disclosure encompasses the methods and techniques, disclosed in the above references.

### Time in Culture

The present disclosure provides method for preparing melanoma cancer stem cells, where the total culturing time including time required for manipulations such as changing media, replating, centrifugation, and sedimenting, is less than 5 months, less than 4 months, less than 3 months, less than 2 months, less than one month, less than 150 days, less than 120 days, less than 90 days, less than 60 days, less than 30 days, or less than 150 days (+/−20 days), less than 120 days (+/−20 days), less than 90 days (+/−20 days), less than 60 days (+/−20 days), less than 30 days (+/−20 days). In exclusionary embodiments, the present disclosure can exclude any method for preparing cancer stem cells, and any population of cancer stem cells prepared by that method, where time required for manipulation is greater than one of the time-frames disclosed above. What is provided is a time in adherent culture, that is indicated by one of the above time-frames. Also, what is a provided is a time in non-adherent culture, that is one of the above time-frames. Moreover, what is also provided is a combined time in adherent culture and in non-adherent culture, that is identified by one of the above time-frames.

### Media of the Present Disclosure.

One formulation variation can use the B27 supplementation with HSA (human serum albumin), while another variant uses the B27 supplementation with BSA (bovine serum albumin). Table 1 discloses the serum free supplement composition is exemplified with human albumin (see the final component in Table 1). In some exemplary implementations, BSA can be used instead of human serum albumin (HSA). Table 11 discloses the components of the medium, RPMI 1640. “PSF” refers to Pen Strep and Fungizone.” PSF is used as an antibiotic and anti-fungal agent. “Neuroblast stem cell media” is described in Table 8 (bovine component in the medium) and in Table 9 (made with human serum albumin; HSA). “NeuroBlast media” and “NeuroBlast Stem Cell media” are one and the same.

The present disclosure encompasses neuron stem cell media, methods for using neuron stem cell medium, cells prepared with the use of neuron stem cell media, methods for administering said cells to subjects, and kits comprising neuron stem cell medium. For each individual component of the neuron stem cell medium, the present disclosure encompasses a range of concentrations that is +/−5% or less, +/−10% or less, +/−15% or less, +/−20% or less, +/−25% or less, +/−30% or less, and the like, with the total volume remaining constant. The present disclosure encompasses neuron stem cell medium, with omission of one or more of the components. The disclosure also encompasses neuron stem cell medium, with one or more substitutions. General examples of substitutions for cell media include, e.g., substituting sodium phosphate for potassium phosphate, sucrose for glycerol, cystine for cysteine, and the like.

### Alternatives to Basic Fibroblast Growth Factor

With culture on non-adherent substrate, medium can optionally include basic fibroblast growth factor (bFGF), bFGF analogue, bFGF in combination with one or more other growth factors, or one or more growth factors with no added bFGF. Growth factors include EHNA compounds (Burton et al (2010) Biochem. Soc. Trans. 38:1058-1061), bone morphogenic protein-2 (BMP-2), vascular endothelial growth factor (VEGF), leukemia inhibitory factor (LIF), insulin growth factor-1 or -2 (IGF-1; IGF-2), transforming growth factor-beta (TGF-beta), and the like.

The present disclosure provides, as an alternative to bFGF, any growth factor or ligand that acts through the MAPK (Mitogen-activated protein kinases). MAPK was originally called ERK (extracellular signal-regulated kinases).

The classic list of growth factors acting through this mechanism includes FGF, EGF, PDGF, NT3/4, BDNF, NGF, VEGF. In addition these also actt the same way: TNF, IL-1, TGFb, FASL. These growth factors act primarily as mitogens.

Another mechanism of fast proliferation that can be exploited is stimulating PI3K-AKT pathway through receptor tyrosine kinases (such as EGF, IGF etc) and GPCRs (G protein-coupled receptors).

In addition to natural ligands that stimulate the above pathways, agents that antagonize the inhibitors are to be considered for in-vitro use for manufacturing. An example of such inhibitor is the PTEN inhibitor that acts in the PI3K-AKT system.

Other cancer treatments use agents against single targets in this pathway. Examples of single targets already used in therapy in vivo include, e.g., Raf kinase inhibitors sorafenib, SB590885, PLX4720, XL281, RAF265, LGX818, vemurafenib; MEK inhibitors: XL518, CI-1040, PD035901, MEK162, selumetinib, Trametinib (GSK1120212).

In contrast to these treatments the present disclosure uses the (agonists) ligands that promote the expansion of the cancer for in vitro manufacturing.

The morphogenic effect of ligands used in the present disclosure (such as FGF, EGF) contributes to preferential expansion of the cancer stem cell population by stimulating transcription factors such as Nanog, cKit, Sox2, Oct3/4 through the enumerated pathways.

One or more of the above natural and synthetic ligands, or any combination thereof, can be added to culture medium during non-adherent culture, e.g., in a low adherent flask, very low adherent flask, or ultra-low adherent flasks, or can be added to culture medium during adherent culture. The following concerns growth in adherent culture. bFGF is not absolutely needed during adherent culture, but it can help maintain the “stem cell” status of the cancer cells by stimulating transcription factors such as Nanog, cKit, Sox2, Oct3/4. Adversely, indiscriminate use or over-use of bFGF may enhance growth of a population that is not tumoral such as normal fibroblasts or epithelial cells. Therefore the use in the adherent stage should be limited in time by assessing the purity of the cancer cell population. The spherogenic step in manufacturing will prevent the expansion of normal cell population and that is the point when growth factors can be used extensively and if impurification is suspected, this step (spherogenic) can be repeated.

Without implying any limitation, the main features of the present disclosure include: (1) Growing in non-adherent conditions, preferably with single growth factors, or combinations of growth factors, or antagonists of inhibitors; (2) Selecting cancer step cells by way of isolating spheroids (and not using any other method to isolate the cancer stem cells). Spheroids can be isolated by gravity methods, centrifugation, filtering, and so on; (3) Growing on adherent conditions, preferably with one or more growth factors; and (4) Optional repeating of the non-adherent process.

TABLE 1
Neural Stem Cell Media

| | Final concentration in medium |
|---|---|
| Component | (mg/L) |
| corticosterone | 0.02 |
| progesterone | 0.005 |
| retinol, all trans | 0.1 |
| retinol acetate | 0.1 |
| insulin | 5 |
| T3 | 0.002 |
| Pluronic F68 | 0.25 |
| lipoic acid | 0.05 |
| tocopherol | 1 |
| tocopherol acetate | 1 |
| linoleic acid | 1 |
| linolenic acid | 1 |
| catalase | 2.5 |
| glutathione reduced | 1 |
| superoxide dismutase | 2.5 |
| l-carnitine | 2 |
| ethanolamine | 1 |
| putrescine | 10 |
| selenium | 0.01 |
| human albumin | 2500 |

TABLE 2
R15-2XPSF (1 L)

| | |
|---|---|
| RPMI-1640 | 800 mL |
| Heat inactivated FBS | 75 mL |
| Iron-supplemented calf | 75 mL |
| serum | |
| L-glutamine | 10 mL |
| Sodium pyruvate | 10 mL |
| 1M HEPES | 10 mL |
| Pen/Strp/Fung | 20 mL |
| (antibiotics) | |

TABLE 3
R15-1XPSF (1 L)
810 mL
75 mL
75 mL
10 mL
10 mL
10 mL
10 mL

TABLE 4
R15 Antibiotic free (1 L)
820 mL
75 mL
75 mL
10 mL
10 mL
10 mL
(zero mL)

TABLE 5
Transport media

| | |
|---|---|
| R15-2XPSF | (amount needed) |
| Gentamicyin (40 mg/mL) | 1.2 microliters per mL R15-2XPSF |

TABLE 6
Serum-free/antibiotic free (1 L)

| | |
|---|---|
| RPMI-1640 | 970 mL |
| L-glutamine | 10 mL |
| Sodium pyruvate | 10 mL |
| 1M HEPES | 10 mL |

TABLE 7
Serum-free 2XPSF (1 L)

| | |
|---|---|
| RPMI-1640 | 950 mL |
| L-glutamine | 10 mL |
| Sodium pyruvate | 10 mL |
| 1M HEPES | 10 mL |
| Pen/Strep/fung | 20 mL |

TABLE 8
Neuroblast with BSA (1 L)

| | |
|---|---|
| KO DMEM/F12 | 970 mL |
| B-27 supplement | 20 mL |
| ITS | 10 mL |
| Glutamax | 10 mL |
| FGF | FGF must be added to a final concentration of 5 |
| | ng/mL or 10 ng/mL right before using. |

TABLE 9
Neuroblast with HSA (1 L)

| | |
|---|---|
| KO DMEM/F12 | 970 mL |
| B-27 supplement | 20 mL |
| ITS | 10 mL |
| Glutamax | 10 mL |
| FGF | FGF must be added to a final concentration of 5 |
| | ng/mL or 10 ng/mL right before using. |

TABLE 10
Cardioblast with B27 HSA based (1 L)

| | |
|---|---|
| KO DMEM/F12 | 970 mL |
| B-27 supplement | 20 mL |
| ITS | 10 mL |
| Glutamax | 10 mL |
| T3 (0.1 mg/mL) | 0.100 mL |
| Insulin | 2 mL of 5 mg/mL stock or 10 micrograms/mL |
| Ascorbic acid | 1 mL of 1000X stock or 20 micrograms/mL |
| FGF | FGF must be added to a final concentration of 10 |
| | ng/mL right before using. |
| EGF | EGF must be added to a final concentration of |
| | 20 ng/mL right before using. |

TABLE 11
RPMI 1640 medium

| | | |
|---|---|---|
| Molecular | Concentration | |
| Weight | (mg/L) | mM |

Amino Acids

| | | | |
|---|---|---|---|
| Glycine | 75 | 10 | 0.133 |
| L-Arginine | 174 | 200 | 1.15 |
| L-Asparagine | 132 | 50 | 0.379 |
| L-Aspartic acid | 133 | 20 | 0.15 |
| L-Cystine 2HCl | 313 | 65 | 0.208 |
| L-Glutamic Acid | 147 | 20 | 0.136 |
| L-Glutamine | 146 | 300 | 2.05 |
| L-Histidine | 155 | 15 | 0.0968 |
| L-Hydroxyproline | 131 | 20 | 0.153 |
| L-lsoleucine | 131 | 50 | 0.382 |
| L-Leucine | 131 | 50 | 0.382 |
| L-Lysine hydrochloride | 183 | 40 | 0.219 |
| L-Methionine | 149 | 15 | 0.101 |
| L-Phenylalanine | 165 | 15 | 0.0909 |
| L-Proline | 115 | 20 | 0.174 |
| L-Serine | 105 | 30 | 0.286 |
| L-Threonine | 119 | 20 | 0.168 |
| L-Tryptophan | 204 | 5 | 0.0245 |
| L-Tyrosine disodium | 261 | 29 | 0.111 |
| salt dihydrate | | | |
| L-Valine | 117 | 20 | 0.171 |

Vitamins

| | | | |
|---|---|---|---|
| Biotin | 244 | 0.2 | 0.00082 |
| Choline chloride | 140 | 3 | 0.0214 |
| D-Calcium pantothenate | 477 | 0.25 | 0.000524 |
| Folic Acid | 441 | 1 | 0.00227 |
| Niacinamide | 122 | 1 | 0.0082 |
| Para-Aminobenzoic Acid | 137 | 1 | 0.0073 |
| Pyridoxine hydrochloride | 206 | 1 | 0.00485 |
| Riboflavin | 376 | 0.2 | 0.000532 |
| Thiamine hydrochloride | 337 | 1 | 0.00297 |
| Vitamin B12 | 1355 | 0.005 | 0.0000037 |
| i-Inositol | 180 | 35 | 0.194 |

Inorganic Salts

| | | | |
|---|---|---|---|
| Calcium nitrate | 236 | 100 | 0.424 |
| (Ca(NO3)2 4H2O) | | | |
| Magnesium Sulfate | 120 | 48.84 | 0.407 |
| (MgSO4) (anhydrous) | | | |
| Potassium Chloride (KCl) | 75 | 400 | 5.33 |
| Sodium Bicarbonate | 84 | 2000 | 23.81 |
| (NaHCO3) | | | |
| Sodium Chloride (NaCl) | 58 | 6000 | 103.45 |
| Sodium Phosphate dibasic | 142 | 800 | 5.63 |
| (Na2HPO4) anhydrous | | | |

Other Components

| | | | |
|---|---|---|---|
| D-Glucose (dextrose) | 180 | 2000 | 11.11 |
| Glutathione (reduced) | 307 | 1 | 0.00326 |
| Phenol Red | 376.4 | 5 | 0.0133 |

### DETAILED DESCRIPTIONS OF THE FIGURES

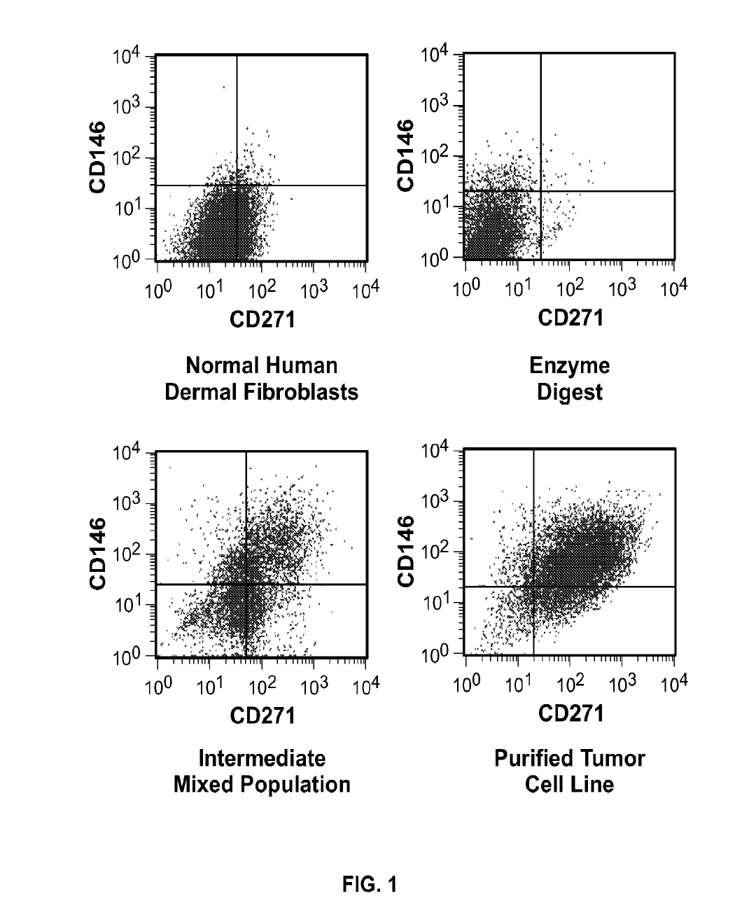

FIG. 1

Melanoma stem cells with neuroendocrine and mesenchymal phenotypes are enriched during the purification process using differential attachment and serum starvation methods. Representative flow cytometry plots for CD146 and CD271 at the beginning of the culture period (Enzyme Digest), at the point of partial purity (Intermediate), and after the purification was complete (Purified) are shown. Normal human dermal fibroblasts (NHDF) were included as a control. Summarized data for eight separately processed samples is shown. Values shown are averages ±SD.

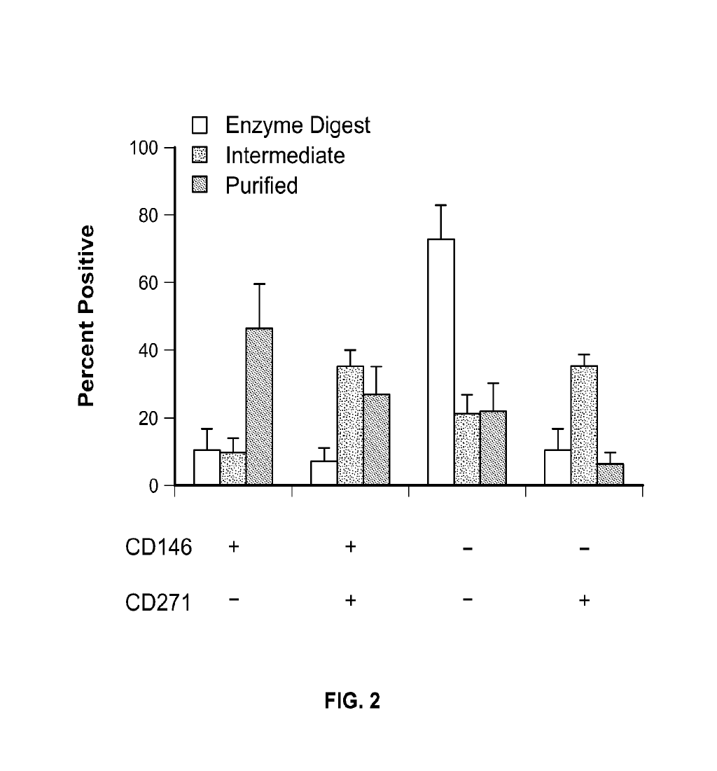

FIG. 2

The histogram discloses the percent of cells expressing CD146 and CD271, for cells at three different stages of preparation. The stages are enzyme digest, intermediate, and purified. Table 12 shows Percentage of expression of CD146 and CD271 in autologous melanoma cell lines used to load dendritic cells for active specific immunotherapy. N=63, for the individual patients comprising the histogram.

TABLE 12

| Patient | CD146+/ | CD146+/ | CD146−/ | CD146−/ |
|---|---|---|---|---|
| Number | CD271− | CD271+ | CD271− | CD271+ |
| 1 | 17 | 82.7 | 0.3 | 0 |
| 2 | 77.3 | 4.1 | 18.5 | 0.1 |
| 3 | 13.3 | 76 | 5.9 | 4.8 |
| 4 | 1.7 | 96.7 | 0.7 | 1 |
| 5 | 33.3 | 64.6 | 1.8 | 0.3 |
| 6 | 32.6 | 67.4 | 0.1 | 0 |
| 7 | 46.5 | 52.7 | 0.8 | 0 |
| 8 | 35.9 | 53.4 | 9.7 | 1.1 |
| 9 | 47.8 | 50.8 | 1.4 | 0.1 |
| 10 | 11 | 14.6 | 47.4 | 27 |
| 11 | 86.8 | 8.2 | 4.9 | 0 |
| 12 | 77.4 | 19.6 | 2.7 | 0.3 |
| 13 | 0.2 | 2.6 | 34 | 63.3 |
| 14 | 0 | 6.7 | 26.1 | 67.3 |
| 15 | 12.6 | 70.9 | 8.3 | 8.2 |
| 16 | 46.4 | 48 | 4.7 | 0.9 |
| 17 | 71 | 20.1 | 8.6 | 0.2 |
| 18 | 59 | 25.9 | 15.1 | 0.1 |
| 19 | 58.1 | 34.4 | 7.2 | 0.3 |
| 20 | 47.3 | 48.5 | 3.8 | 0.4 |
| 21 | 64.1 | 22 | 13.3 | 0.5 |
| 22 | 57.3 | 38.6 | 4.1 | 0.1 |
| 23 | 56 | 43.5 | 0.5 | 0 |
| 24 | 48.4 | 43.1 | 8 | 0.6 |
| 25 | 18.8 | 76.8 | 2.4 | 3 |
| 26 | 27.8 | 40.7 | 30.1 | 1.4 |
| 27 | 79.2 | 9.7 | 11.1 | 0 |
| 28 | 23.5 | 20.3 | 42.4 | 13.8 |
| 29 | 55.9 | 41.8 | 2.2 | 0.1 |
| 30 | 76.4 | 11.9 | 11.7 | 0 |
| 31 | 24.5 | 58.1 | 12.2 | 5.2 |
| 32 | 31.7 | 45.9 | 18.7 | 3.8 |
| 33 | 0.3 | 0.9 | 89.8 | 9 |
| 34 | 79.2 | 10.5 | 10.3 | 0.1 |
| 35 | 47.3 | 50.5 | 2 | 0.3 |
| 36 | 64.2 | 18 | 17.2 | 0.3 |
| 37 | 21.4 | 25.7 | 45.5 | 7.4 |
| 38 | 0.2 | 57.8 | 1.6 | 40.4 |
| 39 | 46.1 | 50.3 | 3.6 | 0 |
| 40 | 46.4 | 52.2 | 1.4 | 0 |
| 41 | 64.2 | 30.9 | 4.8 | 0.1 |
| 42 | 0.3 | 0.5 | 67 | 32.2 |
| 43 | 58.5 | 35.9 | 5.6 | 0 |
| 44 | 30.1 | 24.4 | 38.4 | 7.1 |
| 45 | 68.6 | 30.2 | 1.1 | 0.1 |
| 46 | 21.4 | 14.7 | 56.6 | 7.4 |
| 47 | 54.3 | 33.4 | 9 | 3.3 |
| 48 | 0.2 | 51.3 | 0.4 | 48.2 |
| 49 | 21.8 | 72.9 | 0.6 | 4.7 |
| 50 | 7.8 | 70.8 | 4.9 | 16.5 |
| 51 | 69.9 | 14.6 | 15.2 | 0.4 |
| 52 | 35.5 | 63.6 | 0.9 | 0 |
| 53 | 50.7 | 48.3 | 0.8 | 0.2 |
| 54 | 59.5 | 22.2 | 15.6 | 2.8 |
| 55 | 14.3 | 2.7 | 78 | 5.1 |
| 56 | 19.6 | 78.2 | 1.4 | 0.8 |
| 57 | 17.2 | 80.3 | 2.4 | 0.1 |
| 58 | 29 | 68.6 | 2.4 | 0.1 |
| 59 | 31.2 | 42.2 | 21.2 | 5.5 |
| 60 | 24.7 | 74.4 | 0.5 | 0.5 |
| 61 | 38.9 | 52.7 | 2.1 | 6.3 |
| 62 | 33.6 | 65 | 1.1 | 0.3 |
| 63 | 53.8 | 18.9 | 20.3 | 7 |
| Average | 38.9 | 40.7 | 14 | 6.5 |
| S.D. | 24.2 | 24.7 | 19.6 | 14.3 |

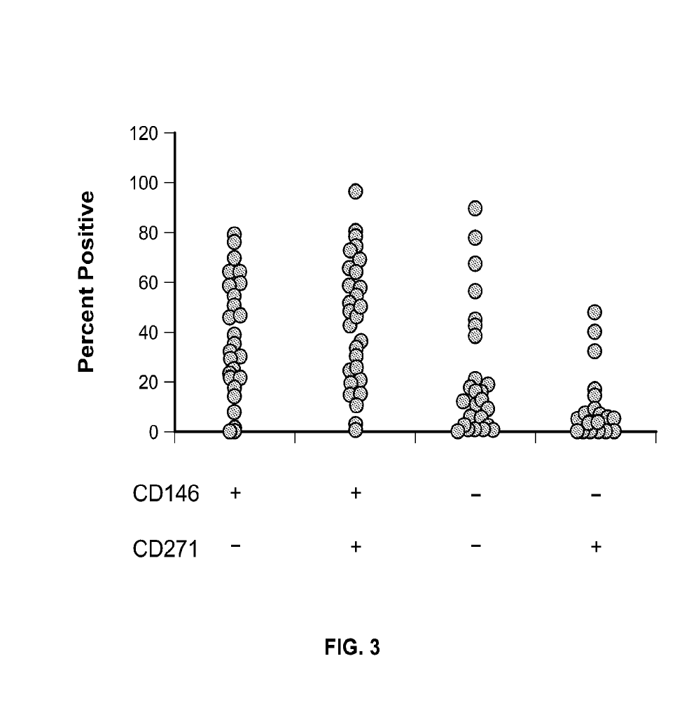

FIG. 3

Autologous melanoma cell lines used to load dendritic cells in active specific immunotherapy contained cells expressing antigens associated with neural crest and mesenchymal origins. Irradiated and cryopreserved purified autologous melanoma cells were assayed by flow cytometry for the expression of CD146 and CD271, N=36, values shown are ±SD.

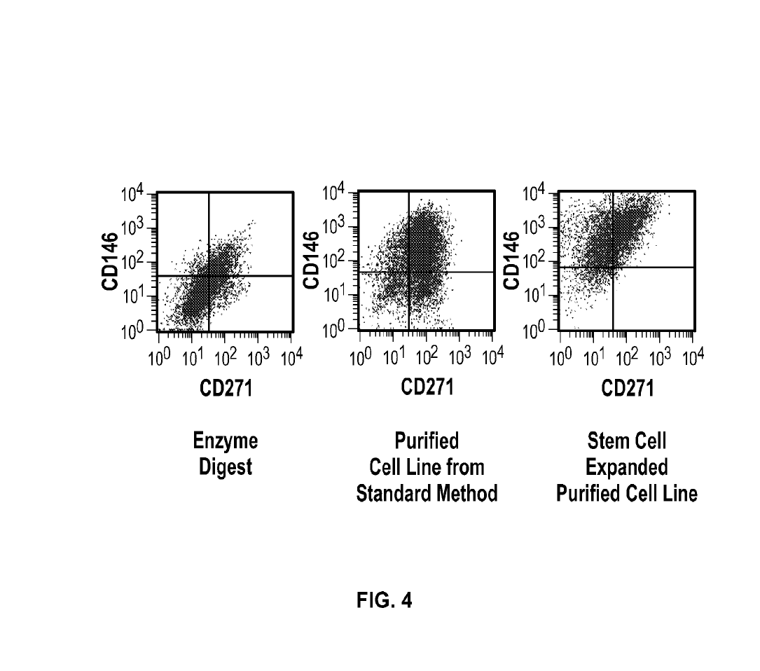

FIG. 4

Melanoma stem cell lines derived from either standard or sphere generating methodologies give rise to cells of the same phenotype. Cells from each of the conditions shown in the figure were assayed by flow cytometry for CD146 and CD271 measuring double positives. FIG. 4 findings as compared to FIG. 3 findings suggest that in some instances there may at least one of a loss of cells that do not form spheres during incubation and an increase in CD271 expression.

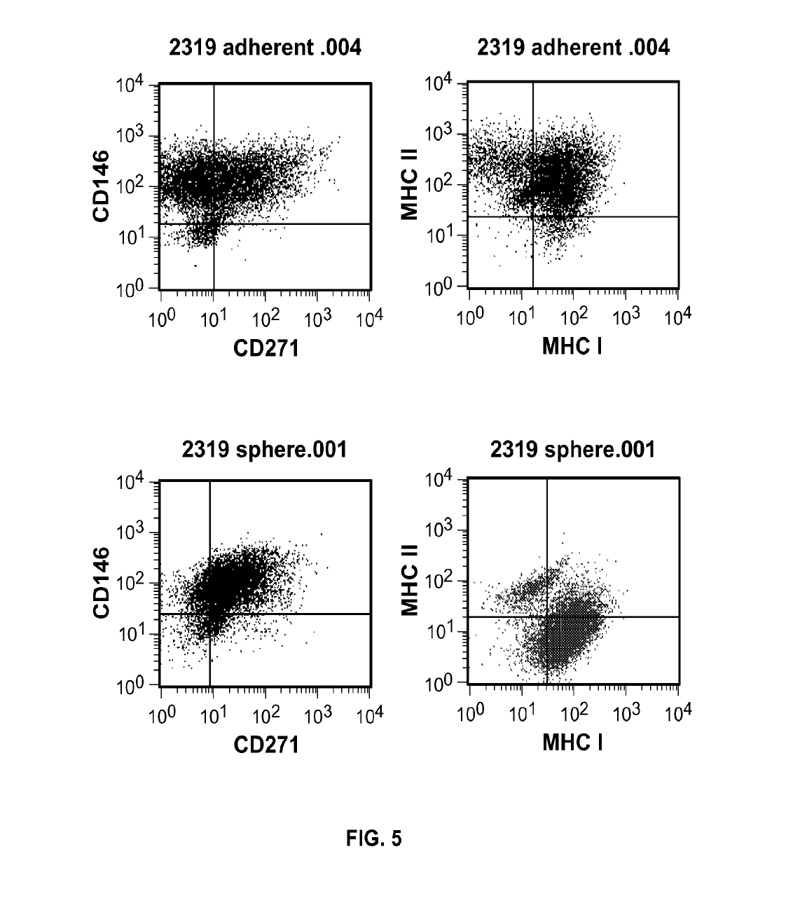

FIG. 5

Purified melanoma cell lines were placed in either neuronal stem cell media or standard serum containing expansion media (15% FBS/RPMI) for period of 7 days. Afterwards, the cells were harvested by trypsinization in the case of the adherent cells and by simple collection of the cancer stem cell spheres. Cells from each of the conditions shown in the figure were simultaneously assayed by flow cytometry for CD146, CD271, MHC class I and MHC class II. Higher MHC II expression stimulates CD4 memory cells which can support and sustain an immune response by secretion of activation cytokines.

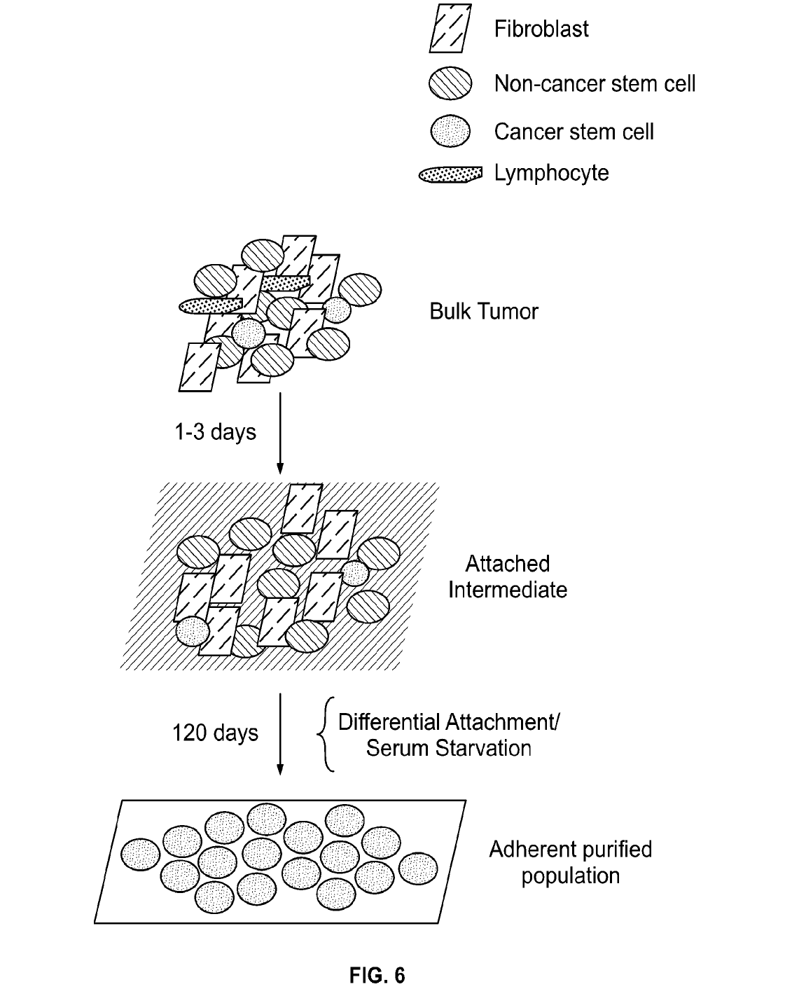

FIG. 6

Schematic representation of the purification of melanoma cancer stem cells process using differential attachment and serum starvation (Method I). Bulk tumor representing enzymatically digested surgical tumor samples are incubated for 1-3 days in serum containing cell culture media then washed twice to remove lymphocytes. The attached mixture of fibroblasts, non-cancer stem cells and cancer stem cells are then subjected to low serum cell culture conditions (range of 1-5% fetal bovine serum) and a series of differential attachment procedures over the course of an average of 120 days. The differential attachment procedure consists of enzymatically detaching the mixture of cells from the substrate and plating onto new substratum (standard plasma treated cell culture flasks) for period of 5-20 minutes until 25-30% of the cell have attached. The non-attached cells are then transferred to a new flask and the attachment procedure repeated for a series of 4-6 times. This process take advantage of the characteristic the higher rate of attachment of fibroblasts compared to that of cancer cells. Additionally, the low serum conditions will inhibit the growth of contaminating fibroblast and non-cancer stem cells growth rates due to higher nutrient requirements of these cells compared to cancer stem cells.

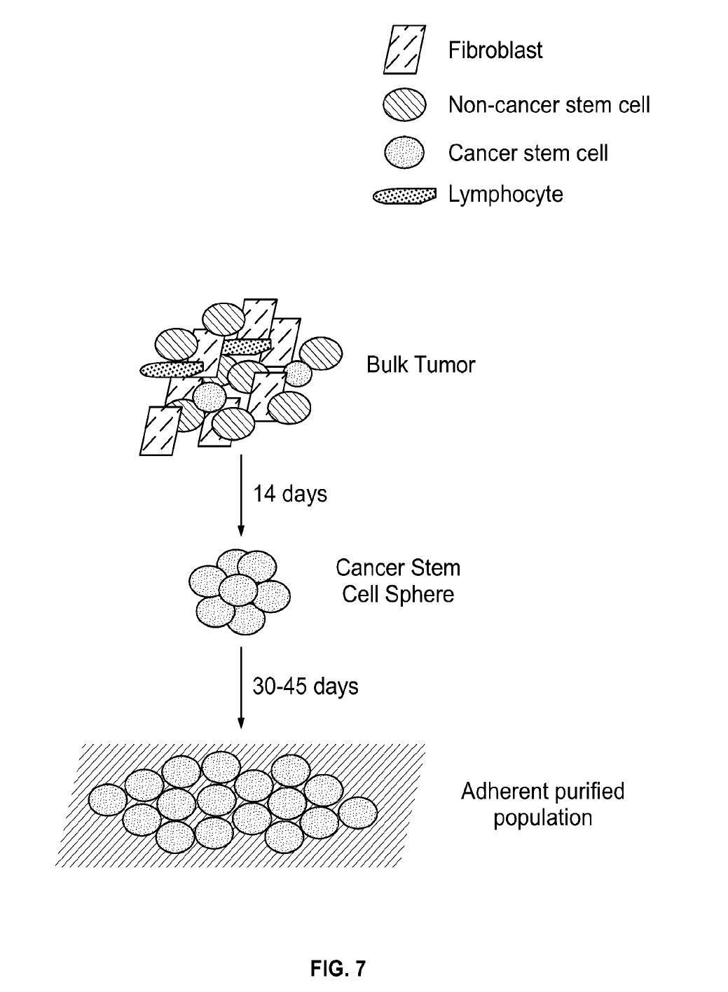

FIG. 7

Schematic representation of purification process using ultra-low adherent stem cell conditions to isolate cancer stem cells followed by adherent expansion conditions (Method 2). Polystyrene coated with a Corning® proprietary material is, in some implementations, used to support ultra-low adherence. Poly-propylene has structure-inherited hydrophobic properties which also will support ultra-low adherence. In addition of Corning® proprietary coatings, a thin agar coating (polysaccharides), polyamides or a siloxane may be used. Bulk tumor representing enzymatically digested surgical tumor samples are incubated in stem cell media under ultra-low adherent or adherent conditions to generated cancer stem cell spheres after 14 days. Adherency refers to cells that remain attached to the surface that they are growing on; non-attached cells will be removed during the washing and harvesting. Non adherent condition refers to culture environment when the cells are not attached to a substrate other than a similar live cell. Hydrophobic materials or non-biofouling treatments can be used to achieve non-adherent conditions: agarose, poly-ethylene, fluoro-polymers, siloxanes. Conditions that can promote non-adherent conditions include lack of serum components or lack of peptides with terminations specific for integrins (RGD, IKVAV, YIGSR, RETTAWA etc) from media or substrates. Also, addition of membrane expanders or tensioactive agents: Pluronic F-68, Tween80, Poly-Vinyl Alcohol (PVA), Poly-Ethylene Glycol (PEG) can promote non-adherent conditions. Hyaluronidase which can act as a mobility enzyme in higher concentrations can cause cell detachment. It may also cause a lack of CD44 dependent anchorage. The skilled artisan in the field of mammalian cell culture can readily distinguish between culture flasks, culture dishes, and other culture containers, that are ultra-low adherent and those that are low adherent.

This results in the removal of contaminating populations of cells such as lymphocytes and fibroblasts and non-cancer stem cell tumor cells. These spheres are enriched for CD146/CD271 positive cancer stem cell populations. The spheres are then dissociated either mechanically or enzymatically and plated onto adherent surfaces and allowed to replicate for a further 30-45 days. The cells are then harvested for use in immunotherapy as either whole cells or lysates.

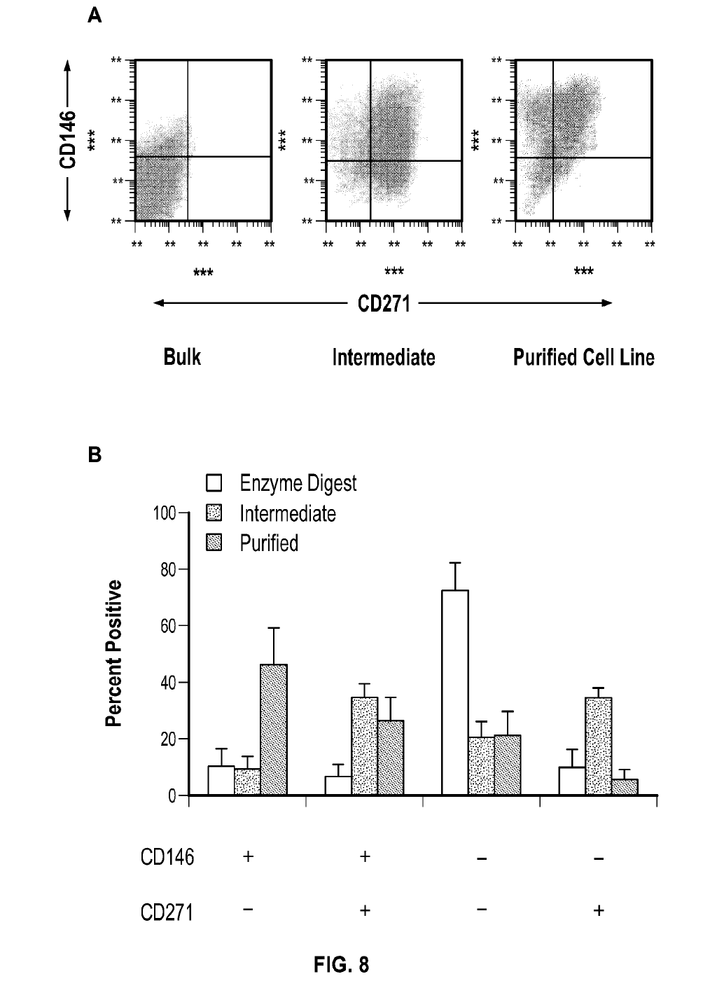

FIG. 8

FIG. 8 discloses enrichment of cancer stem cells during purification process. FIG. 8A shows flow cytometry results, and FIG. 8B shows a historgram that summarizes flow cytometry results. The results demonstrate that, for this particular cell preparation, in the enzyme digest (bulk tumor, open bars) the most prevalent cell type is CD146 minus/CD271 minus, and that at a purified step of the present disclosure, the most prevalent cell type is CD146 plus/CD271 minus. At the intermediate stage, CD146 plus/CD271 cells and CD146 minus/CD271 plus cells occurred in roughly equal percentages.

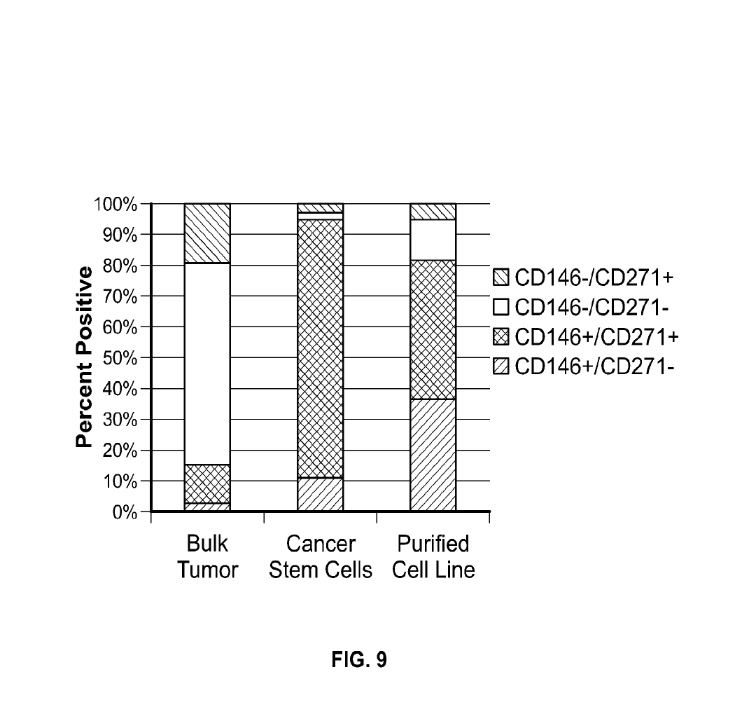

FIG. 9

FIG. 9 discloses expression of CD146 and CD271 in bulk tumor cells, in “cancer stem cells” of the present disclosure, and in “purified cell line” produced by a standard method.

Regarding FIG. 9, in bulk tumor cells, the CD146 minus/CD271 minus cells (open segment of bar) account for the greatest proportion of cells, with CD146 minus/CD271 plus cells (downwards-slanted hatches in segment of bar) accounting for the next-most prevalent cells. In “cancer stem cells,” the CD146 plus/CD271 plus cells (criss-cross hatches in segment of bar) is the most prevalent type of cell. In the “purified cell line, CD146 plus/CD271 plus cells (criss-cross hatches in segment of bar) and the CD146 plus/CD271 minus cells (upwards-slanted hatches in segment of bar) are roughly equivalent in proportion, with the CD146 plus/CD271 plus cells occurring at a somewhat greater percentage than the CD146 plus/CD271 minus cells. FIG. 9 represents the data from Table 13.

TABLE 13
Data used for histogram of FIG. 9

| | Days in | | CD146plus/ | CD146plus/ | CD146minus/ | CD146minus/ |
|---|---|---|---|---|---|---|
| | culture | Yield ×10\hat{ }6 | CD271minus | CD271plus | CD271minus | CD271plus |
| Cancer Stem | | | | | | |
| Cell Derived | | | | | | |
| 2352 | 39 | 125 | 3.04 | 93.39 | 0.55 | 3.02 |
| 2350 | 39 | 280 | 2.93 | 92.45 | 1.25 | 3.37 |
| 2266 | 39 | 92 | 14.52 | 77.84 | 5.7 | 2.44 |
| Standard | | | | | | |
| Method Cell | | | | | | |
| Lines | | | | | | |
| 2352 | 57 | 150 | 55.86 | 36.57 | 7.02 | 0.55 |
| 2350 | 76 | 200 | 19.95 | 61.73 | 9.52 | 8.8 |

| | | | |
|---|---|---|---|
| 2266 | NA | NA | (not a previously established cell line) |

### Examples

This present disclosure provides methods and reagents for isolating and expanding cancer stem cells of mesenchymal and neural crest origin from biopsies of melanoma samples for use in cell-based immunotherapy. Methodologies include the use of media formulations to isolate and then proliferate distinct populations of tumor stem cells with a neural crest and/or mesenchymal phenotype. CD146 is a marker frequently found on mesenchymal cells and is associated with highly invasive phenotype. CD271, neuronal growth factor receptor p75, is expressed by neuronal precursor cells and is expressed on melanoma initiating cells. What is provided are steps for isolating and expanding a type of cells that can be found in metastatic melanoma preparations where by these cells are either CD146+/CD271−, CD146+/CD271+, or CD146−/CD271+. In addition, these cells may also be positive for CD44, Twist, Zeb1/2, Snail, Slug, SIP, CD133, CD166, CXCR4, Notch-1 and CD90 in total or in part. The cells are cultivated under non-adherent conditions for 10-14 days in stem cell media and then switched to adherent conditions under non-stem cell expansion media. The final adherent step is to promote the up-regulation of major histocompatibility complexes and down-regulation of immunosuppressive molecules like indoleamine-pyrrole 2,3-dioxygenase, tumor growth factor beta and interleukin-10. The present disclosure uses these cells to immunize patients rather than bulk tumor biopsies or purified non-adherent cancer stem cells. Thus, the present disclosure uses cells that are identified as tumor forming as opposed to differentiated, non-proliferating cell populations which are the predominant cell in a bulk tumor biopsy or immunosuppressive cancer stem cells that are still in the non-adherent “spheroid” state. Selvan et al (2010) Melanoma Res. 20:280-292, disclose reagents and methods for processing melanoma tissue samples.

Cell-based immunotherapy is expected to be effective in the autologous setting due to presence of tumor-associated neo-antigens. However, the use of bulk autologous tumor preparations have not yielded the expected clinical results likely due to the fact that population of cells present in bulk tumor are mainly differentiated cells with a low percentage of cells representing cancer stem cells. The techniques of the present disclosure demonstrate that tumor specimens must be processed in a manner that enriches for cancer stem cells to be most effective. In particular, the process results in the enrichment of cells that are either CD146+/CD271− (mesenchymal cancer cell), or CD271/CD146 (cancer stem cell with mesenchymal characteristics), using the sphere forming technique.

However, the cells must be then be attached to a substrate such as cell culture flask for the final proliferation steps before use in immunotherapy to guard against the immunosuppressive effects of cancer stem cells (Wei et al (2010) Glioma-associated cancer-initiating cells induce immunosuppression. Clin Cancer Res. 16:461-473; Schatton et al (2010) Modulation of T-cell activation by malignant melanoma initiating cells. Cancer Res. 70:697-708).

The effect of expanding the cells on an adherent substrate such as a standard cell culture flask or similar surface is to up-regulate important immune associated proteins called major histocompatibility complexes (MHC class I and class II). These protein complexes are primary mechanism by which the immune system recognizes and responses to cells that are either foreign or infected with viruses. Cancer stem cells down regulate those molecules while in the detached, spheroid phase of growth and up-regulate them while in the attached, expanding phase of growth. The act of transferring the cancer stem cells from a non-adherent to an adherent state during expansion is expected to reduce or eliminate the ability of cancer stem cells to suppress an immune response. In an addition to MHC class I and class II proteins, cancer stem cells also up-regulate other immunosuppressive molecules such as transforming growth factor-beta (TGF-b), indoleamine-pyrrole 2,3-dioxygenase (IDO) and interleukin-10 (IL-10) (Jewett, A. and H. C. Tseng, Tumor induced inactivation of natural killer cell cytotoxic function; implication in growth, expansion and differentiation of cancer stem cells. J. Cancer, 2011. 2:443-457). Subsequently, these factors should be down-regulated in response to adherence to a substrate.

### Percent Down-Regulation and Percent Up-Regulation

The present disclosure provides cells, and related methods and compositions, wherein the culture on an adherent surface results in down-regulation of an immunosuppressive molecule, and (i) wherein the immunosuppressive molecule is at least one of indoleamine-pyrrole-2,3-dioxygenase, tumor growth factor-beta, and interleukin-10 (IL-10), and (ii) wherein the expression of the at least one immunosuppressive molecule prior to culture on adherent surface is 100%, and wherein down-regulation after culture on the adherent surface results in an expression that is at a level that is less than 80%, less than 70%, less than 60%, less than 50%, less than 40%, less than 30%, as compared to the initial 100%.

Also, the present disclosure provides cells, and related methods and compositions, wherein the culture on an adherent surface results in down-regulation of an immunosuppressive molecule, and (i) wherein the immunosuppressive molecule is indoleamine-pyrrole-2,3-dioxygenase and (ii) wherein the expression of the immunosuppressive molecule prior to culture on adherent surface is 100%, and wherein down-regulation after culture on the adherent surface results in an expression that is at a level that is less than 80%, less than 70%, less than 60%, less than 50%, less than 40%, less than 30%, as compared to the initial 100%.

What is also provided is cells, and related methods and compositions, wherein the culture on an adherent surface results in down-regulation of an immunosuppressive molecule, and (i) wherein the immunosuppressive molecule is tumor growth factor-beta, and (ii) wherein the expression of the immunosuppressive molecule prior to culture on adherent surface is 100%, and wherein down-regulation after culture on the adherent surface results in an expression that is at a level that is less than 80%, less than 70%, less than 60%, less than 50%, less than 40%, less than 30%, as compared to the initial 100%.

In another aspect, what is provided is cells, and related methods and compositions, wherein the culture on an adherent surface results in down-regulation of an immunosuppressive molecule, and (i) wherein the immunosuppressive molecule is interleukin-10 (IL-10), and (ii) wherein the expression of the immunosuppressive molecule prior to culture on adherent surface is 100%, and wherein down-regulation after culture on the adherent surface results in an expression that is at a level that is less than 80%, less than 70%, less than 60%, less than 50%, less than 40%, less than 30%, as compared to the initial 100%.

In embodiments, the up-regulation of a particular nucleic acid or polypeptide is detectable in at least 20% of a cell population, in at least 30%, in at least 40%, in at least 50%, in at least 60%, in at least 70%, in at least 80%, in at least 90%, of cell population. Regarding down-regulation, the down-regulation of a particular nucleic acid or polypeptide is detectable in at least 20% of a cell population, in at least 30%, in at least 40%, in at least 50%, in at least 60%, in at least 70%, in at least 80%, in at least 90%, of a cell population.

In embodiments, the up-regulation of a particular nucleic acid or polypeptide is detectable in at least 20% of a population of cancer stem cells, in at least 30%, in at least 40%, in at least 50%, in at least 60%, in at least 70%, in at least 80%, in at least 90%, of a population of cancer stem cells. Regarding down-regulation, the down-regulation of a particular nucleic acid or polypeptide is detectable in at least 20% of cell population, in at least 30%, in at least 40%, in at least 50%, in at least 60%, in at least 70%, in at least 80%, in at least 90%, of a population of cancer stem cells.

The related methods and compositions, mentioned above, encompass methods of cell culture, methods for loading cancer stem cells on dendritic cells (DCs), methods for preparing a vaccine, a composition that is a vaccine comprising DCs loaded with melanoma cancer stem cells, methods for administering the vaccine to a subject, to a subject at risk for melanoma, or to a subject that comprises melanoma, and methods for stimulating specific immune response against at least one melanoma-specific antigen, methods for improving an objective endpoint as measurable by RECIST criteria, and methods for improving a clinical endpoint, such as progression-free survival (PFS), time to distant metastasis (TDM), or overall survival (OS).

The present disclosure provides cells, and related methods and compositions, wherein the culture on an adherent surface results in up-regulation of MHC-I, of MHC-II, or of both MHC-I and MHC-II, and (ii) wherein the expression of the MHC-I, of MHC-II, or of both MHC-I and MHC-II, prior to culture on adherent surface is 100%, and wherein up-regulation after culture on the adherent surface results in an expression that is at a level that is at least 125%, at least 150%, at least 200% (2-fold increase), at least 250%, at least 300%, at least 400% (4-fold increase), at least 500%, as compared to the initial 100%. MHC is major histocompatibility complex. Methods are available for measuring expression of MHC Class I, or of MHC Class II, and for quantifying the expression as up-regulation or as down-regulation (see, e.g., Pantel et al (1991) Cancer Res. 51:4712-4715; Vertuani et al (2009) Cancer Immunol. Immunother. 58:653-664; Yadav et al (2009) J. Immunol. 182:39-43; Lollini et al (1998) Int. J. Cancer. 77:937-941).

A variety of non-limiting methods for detecting expression or for detecting up-regulation of indoleamine-pyrrole-2,3-dioxygenase (Orabona et al (2006) Blood. 107:2846-2854, interleukin-10 (IL-10) (Hedrich and Bream (2010) Immunol. Res. 47:185-206), and tumor growth factor-beta (Kloen et al (1997) Cancer. 80:2230-2239), are cited.

The present disclosure provides prepared melanoma cells, provides dendritic cells loaded the prepared melanoma cells, and provides vaccine comprising provides dendritic cells loaded the prepared melanoma cells, wherein immunosuppression is reduced to less than 90% maximal immunosuppression, to less than 85%, to less than 80%, to less than 75%, to less than 70%, to less than 60%, to less than 50%, to less than 40%, to less than 30%, and the like, of the maximal immunosuppression. In this context, “immunosuppression” refers to any immunosuppressive (tolerizing) ability of one or more melanoma antigens, or to a vaccine comprising dendritic cells loaded with purified melanoma antigens, or to a vaccine comprising dendritic cells loaded with processed melanoma cells, that is, where tolerance is raised against one or more melanoma-specific tumor antigen. Without implying any limitation, a vaccine of the present disclosure can comprise dendritic cells (DCs) loaded with spheres, loaded with a population of cells that comprises spheres, loaded with a population of cells that was derived from spheres and that were expanded on an adherent surface prior to loading on DCs, loaded with spheres that were subjected to homogenization or sonication prior to loading on DCs, loaded with a population of expanded cells that were subjected to homogenization or sonication prior to loading on DCs, and so on.

Enrichment of tumor stem cells with mesenchymal characteristics by in vitro cell culture techniques would make it possible to use these cells in a cell-based immunotherapy protocol. These methods can be used on samples from breast, glioblastoma, mesothelioma, ovarian, lung, prostate, liver and colon cancer biopsies.

Table 14 discloses the expression of common melanoma associate antigens on cell lines derived from standard methodology of differential attachment and serum starvation. See also, e.g., Selvan et al (2008) Int. J. Cancer. 122:1374-1383; Selvan et al (2010) Melanoma Res. 20:280-292.

TABLE 14
Expression of the indicated antigens in a selection
of 94 melanoma cell lines. The number 94 represents
94 separate cell lines from 94 different patients.

| | Expression level |
|---|---|
| Antigens | (positive cell lines/total cell lines) |
| S100 | 28/94* (30%) |
| HMB45 (gp-100) | 71/94 (76%) |
| Melan-A/Mart-1 | 77/94 (82%) |
| MAGE-1 | 47/94 (50%) |
| Tyrosinase | 81/94 (86%) |
| Mel-5 (TRP-1) | 60/94 (64%) |
| HLA-1 | 91/94 (97%) |
| HLA-2 | 78/94 (83%) |
| Fibroblast (1-25%) | 4/94 (4%) |

In exemplary implementations, the present disclosure provides an isolated population of cancer stem cells where at least 20%, at least 40%, at least 50%, at least 60%, at least 70%, at least 80%, at least 90%, at least 95%, at least 98%, of the cells are CD146+/CD271−, where about 0%, about 5%, about 10%, about 20%, about 40%, about 60%, about 80%, about 90%, or at least 10%, at least 20%, at least 30%, at least 40%, at least 50%, at least 60%, at least 70%, at least 80%, at least 90%, or at least 95%, of the same cells express one or more of CD44, Twist, Zeb1/2, Snail, Slug, SIP, CD133, CD166, CXCR4, Notch-1, and CD90.

In exemplary implementations, the present disclosure provides an isolated population of cancer stem cells where at least 20%, at least 40%, at least 50%, at least 60%, at least 70%, at least 80%, at least 90%, at least 95%, at least 98%, of the cells are CD146−/CD271+, where about 0%, about 5%, about 10%, about 20%, about 40%, about 60%, about 80%, about 90%, or at least 10%, at least 20%, at least 30%, at least 40%, at least 50%, at least 60%, at least 70%, at least 80%, at least 90%, or at least 95%, of the same cells express one or more of CD44, Twist, Zeb1/2, Snail, Slug, SIP, CD133, CD166, CXCR4, Notch-1, and CD90.

In exemplary implementations, the present disclosure provides an isolated population of cancer stem cells where at least 20%, at least 40%, at least 50%, at least 60%, at least 70%, at least 80%, at least 90%, at least 95%, at least 98%, of the cells are CD146+/CD271+, where about 0%, about 5%, about 10%, about 20%, about 40%, about 60%, about 80%, about 90%, or at least 10%, at least 20%, at least 30%, at least 40%, at least 50%, at least 60%, at least 70%, at least 80%, at least 90%, or at least 95%, of the same cells express one or more of CD44, Twist, Zeb1/2, Snail, Slug, SIP, CD133, CD166, CXCR4, Notch-1, and CD90.

In exemplary implementations, the isolated population of cancer stem cells is about 100 cells, about 1,000 cells, about 2,000 cells, about 5,000 cells, about 10,000 cells, about 20,000 cells, about 50,000 cells, about 100,000 cells, about 200,000 cells, about 500,000 cells, about 1×10⁶ cells, about 2×10⁶ cells, about 5×10⁶ cells, about 10×10⁶ cells, about 20×10⁶ cells, about 50×10⁶ cells, about 100×10⁶ cells, about 200×10⁶ cells, about 500×10⁶ cells, about 1×10⁹ cells, about 2×10⁹ cells, about 5×10⁹ cells, about 10×10⁹ cells, about 20×10⁹ cells, about 50×10⁹ cells, about 100×10⁹ cells, and the like.

### Methods

### Method 1. Standard Methodology for Generating Melanoma Cancer Cell Lines

The methods used to generate purified tumor cells lines used in the referenced clinical trials were differential attachment and serum starvation whereby fibroblasts and normal stromal cells are eliminated (Dillman et al (1993) Establishing in vitro cultures of autologous tumor cells for use in active specific immunotherapy. J. Immunother. Emphasis Tumor Immunol. 14:65-69).

Surgical specimens of about at least a few hundred cells were obtained after pathological examination and processed into single-cell suspension by mincing with scalpels and collagenase digestion (enzyme digest). The resulting purified cell cultures were expanded to approximately 200 million cells and irradiated prior to cryopreservation and storage in liquid nitrogen. Eligible patients under went apheresis to obtain monocytes for purification by elutriation. Purified monocytes were then differentiated into dendritic cells using 178 ng/mL GM-CSF and 80 ng/mL IL-4 (Cell Genetics) in AIMV (Invitrogen). The resulting dendritic cells were then antigen loaded with irradiated purified tumor cells (DC+TC). Patients received eight sub-cutaneous injections of DC+TC resuspended in 500 micrograms of GM-CSF. Those of ordinary skill in the art, will understand that it is within the scope of this disclosure that in some instances it may be possible to augment or replace (in whole or in part) GM-CSF with at least one of a TLR agonist and CD40 ligand.

Expression of a panel of antigens expressed in the melanoma lines were determined using immunocytochemical procedure. Briefly, cells were cultured in 8-chamber culture slides (Thermo Fisher) in the presence or absence of 1000 IU/mL IFN-c. After 72 hours, the cells were washed three times with phosphate buffered saline (PBS) and fixed in cold acetone. After blocking endogenous peroxidase, the cells were incubated with appropriate primary antibodies against the antigens listed. Immunohistochemistry was performed using biotinylated anti-mouse or rabbit immunoglobulins, Super Sensitive enzyme-conjugated streptavidin labeling and horse radish peroxidase chromogen, and substrate kits (Biogenex, Fremont, Calif.). The reactivity of the following anti-human polyclonal or mono-clonal antibodies was investigated with isotype-matched control antibody: S-100 and HMB-45 (Biogenex), Mel-2, Mel-5, Mart-1 (Signet), Tyrosinase, Mage-1 (Thermo Scientific, Waltham, Mass.), Melan-A, HLA class I, and HLA class II (Dako, Carpinteria, Calif.).

### Method 2. Cancer Stem Cell Spheroid Method of Purification of Cancer Cell Lines

Surgical tumor samples were processed by mincing with scalpels and collagenase digestion. The resulting cell suspensions were placed in neuron stem cell media (Neuroblast stem cell media, California Stem Cell, Irvine, Calif.) at 0.05-0.2 million cells/mL for 21 days in ultra-low adherent cell culture flasks (Corning). During culture on ultra-low adherent substrate, bFGF is not absolutely needed, however, bFGF promotes a more rapid proliferation. The present disclosure provides populations of cells, populations of spheres, and related methods, where bFGF was not used during culture on ultra-low adherent substrate, and also where bFGF was in fact included during culture on ultra-low adherent substrate. The tumor stem cell spheres were recovered using sedimentation and re-cultured in fresh medium every two-three days. After the 21 day spheroid culture period, the spheres are dissociated by enzymatic trypsinization to yield a single celled suspension. The cells are then placed in standard cell culture flasks (Corning, Corning, N.Y.) in RPMI medium containing 15% fetal bovine serum or animal product-free expansion media (Omega Scientific, Tarzana, Calif.) and expanded to establish proliferating adherent cell cultures. Other expansion media formulation can be used that provide for adequate nutrients to ensure rapid expansion of the adherent population of cancer cells.

Purified tumor cell culture, cancer stem cell sphere, and enzyme digest samples were assayed for the expression of one or more of following by flow cytometry after thawing from cryopreservation or during cell culturing: MHC class I, MHC class II and CD146 and CD271 (BD Biosciences, San Jose, Calif.). In addition, control samples of normal human dermal fibroblasts were also assayed. Cells were fixed in 4% paraformaldehyde (Sigma-Aldrich, St. Louis, Mo.) for 15 minutes, washed twice with phosphate buffered saline (PBS) (Omega Scientific) and re-suspended at 1 million cells per milliliter. The cells were stained with 10 uL of CD146 and CD271 or isotype control (BD Biosciences) for 30 minutes, washed with PBS and flow cytometry conducted as per manufacturing instructions on a bead-calibrating FACS Calibur (BD Biosciences).

In cell expansion exemplary implementations, cells subjected to procedure is about 1 cell, exactly 1 cell, about 10 cells, about 20 cells, about 50 cells, 100 cells, about 1,000 cells, about 2,000 cells, about 5,000 cells, about 10,000 cells, about 20,000 cells, about 50,000 cells, about 100,000 cells, about 200,000 cells, about 500,000 cells, about 1×10⁶ cells, about 2×10⁶ cells, about 5×10⁶ cells, about 10×10⁶ cells, about 20×10⁶ cells, about 50×10⁶ cells, about 100×10⁶ cells, about 200×10⁶ cells, about 500×10⁶ cells, about 1×10⁹ cells, about 2×10⁹ cells, about 5×10⁹ cells, about 10×10⁹ cells, about 20×10⁹ cells, about 50×10⁹ cells, about 100×10⁹ cells, and the like.

In some instances it may be useful to add one or more of the following steps to Method 2. An adherence step onto plasma treated tissue culture flasks substantially immediately following enzyme digestion of the tumor followed by a washing step to remove lymphocytes and debris that are not attached to the flask. An incubation step, wherein the washing step may be followed by incubation of the adhered mixture of cancer cells and normal cells in neuronal stem cell media which will produce budding of cancer stem cell spheres from the surface of the flask. A collection step wherein the budding cancer stem cells may be collected and placed in ultra-low adherent conditions for further propagation.

### Modified Method 2. Cancer Stem Cell Spheroid Method of Purification of Cancer Cell Lines

The following non-limiting protocol is for processing and characterizing tumor cell lines that are generated by microsphere technique. Microsphere technique is disclosed below.

Step 1. Randomly select eight enzymatically digested melanoma tumor samples.

Step 2. Thaw cryovials in a water bath set at 37 degrees C., resulting in a cell suspension. Add the suspension dropwise to 15 mL conical tubes containing 5% RPMI medium.

Step 3. Centrifuge at 1200 rpm for 5 minutes.

Step 4. Resuspend in 10 mL of Neuroblast media.

Step 5. Perform a cell count and viability test using a hemocytometer.

Step 6. Resuspend at 80,000 viable cells/mL in Neuroplast plus 10 ng/mL bFGF and place in ultra-low adherent flasks at 0.52 ml per square centimeter.

Step 7. Every 2-3 days, centrifuge cells at 900 rpm for 5 min, and replace with fresh media. Repeat this for the first three media changes, then switch to passive sedimentation for the remaining culture period for a total of 21 days. Passive sedimentation consists of transferring the cell suspension to a 50 mL conical tube, and placing the conical tube in a holder on a flat surface for 3-5 minutes. Observe for collection of microspheres at the bottom of the conical tube. Remove the supernatant and resuspend the cell pellet in Neuroblast plus 5% FBS supplemented with 10 ng/mL bFGF. At the end of 21 days, perform the passive sedimentation.

Step 8. Remove the supernatant, and dissociated the cell pellet with TrypLE for 10 min with gentle pipetting. Perform a cell count and assess viability using a hemocytometer.

Step 9. Resuspend the cells at 20,000 to 30,000 viable cells per square centimeter in NeuroBlast plus 5% FBS supplemented with 10 ng/mL bFGF in standard adherent cell culture flasks. Maintain the cell cultures for 3-4 weeks while changing the media 2-3 times per week, depending on media usage. Take phase contrast photographs periodically.

Step 10. At the end of the expansion period, passage the cells with TrypLE and perform cell counts.

Step 11. Samples from the prepared cells can be characterized as follows. Fix 3-5 million cells by incubating in paraformaldehyde fixation, for flow cytometry characterization using antibodies against CD146 and CD271. Cells were also stained with either isotype IgG1-PE and IgG1-FITC, CD146-PE and CD271-FITC labeled antibodies for 30 minutes in the dark at room temperature in PBS. The stained cells are centrifuged at 400×g for 5 minutes and washed once with PBS. The cells are then resuspended in 0.4 mL of PBS and used for flow cytometry on a BD FACSCalibur® instrument.

### Administration to Subjects

The dendritic cell vaccine is administered subcutaneously (SC). Each dose ranges from 5-20 million loaded DCs, repeated in a series of 8 doses. The injections (4) are given every week for the first month, and every month after the next 4 injections. In alternative exemplary implementations, administration is once a week for 3 weeks then once a month for 5 months for a total of 8 weeks. In some exemplary implementations, a boost adjuvant (GM-CSF) is given simultaneously with every dose. In alternative exemplary implementations, GM-CSF boost adjuvant is given, but not with every single dose. In other exemplary implementations, there is no GM-CSF boost adjuvant at all.

Without limitation, dendritic cells (e.g., autologous or allogeneic dendritic cells) are contacted with cancer stem cell antigens as a cell lysate, acid elution, cell extract, partially purified antigens, purified antigens, isolated antigens, partially purified peptides, purified peptides, isolated peptides, synthetic peptides, or any combination thereof. The dendritic cells are then administered to a subject, for example, a subject comprising a cancer, or a control subject not comprising a cancer. In exemplary implementations, dendritic cells are contacted with, injected into, or administered, by one or more of a route that is subcutaneous, intranodal, intramuscular, intravenous, intranasal, inhaled, oral, by application to intestinal lumen, and the like (see, e.g., O'Neill et al (2004) Blood. 104:2235-2246; Sabado and Bhardwaj (2010) Immunotherapy. 2:37-56).

Thus, while there have shown and described and pointed out fundamental novel features of the disclosure as applied to exemplary implementations and/or aspects thereof, it will be understood that various omissions, reconfigurations and substitutions, and changes in the form and details of the exemplary implementations, disclosure and aspects thereof, may be made by those skilled in the art without departing from the spirit of the disclosure and/or claims. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or implementation may be incorporated in any other disclosed or described or suggested form or implementation as a general matter of design choice. It is the intention, therefore, to not limit the scope of the disclosure. All such modifications are intended to be within the scope of the claims appended hereto.

All publications, patents, patent applications and references cited in this specification are herein incorporated by this reference as if fully set forth herein.

While method and apparatus have been described in terms of what are presently considered to be the most practical and preferred implementation, exemplars and/or embodiments, it is to be understood that the disclosure need not be limited to the disclosed implementation, exemplars and/or embodiments. It is intended to cover various modifications and similar arrangements included within the spirit and scope of the claims, the scope of which should be accorded the broadest interpretation so as to encompass all such modifications and similar structures.

It should also be understood that a variety of changes may be made without departing from the essence of the invention. Such changes are also implicitly included in the description. They still fall within the scope of this disclosure. It should be understood that this disclosure is intended to yield a patent covering numerous aspects both independently and as an overall system and in both method and apparatus modes.

Further, each of the various elements of the disclosure, exemplars, aspects thereof and claims may also be achieved in a variety of manners. This disclosure should be understood to encompass each such variation, be it a variation of any apparatus, a method or process, or even merely a variation of any element of these.

Particularly, it should be understood that as the disclosure relates to elements claimed, the words for each element may be expressed by equivalent apparatus terms or method terms—even if only the function or result is the same.

Such equivalent, brooder, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference.

Finally, all references listed in the Information Disclosure Statement or other information statement filed with the application or thereafter are hereby appended and hereby incorporated by reference; however, as to each of the above, to the extent that such information or statements incorporated by reference might be considered inconsistent with the patenting of claimed invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to 35 USC §132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

The Abstract is provided to comply with 37 CFR §1.72(b) to allow the reader to quickly ascertain the nature and gist of the technical disclosure. The Abstract is submitted with the understanding that it will not be used to interpret or limit the scope or meaning of the claims.

1-25. (canceled)

26. A method for producing purified adherent melanoma cancer stem cells, comprising the steps of:
(a) obtaining a tissue specimen from a biopsy of a melanoma tumor from a subject;
(b) dissociating the tissue specimen mechanically;
(c) enzymatically dissociating samples of the tissue specimen into a single-cell suspension
(d) immersing the single-cell suspension of (c) in serum free cell culture media comprising basic fibroblast growth factor (bFGF) and culturing on a low adherent or an ultra-low adherent surface to produce a cancer cell culture comprising melanoma cancer cell spheroids;
(e) sedimenting the spheroids to collect microspheres;
(f) dissociating cells from the microspheres to yield a single-cell suspension;
(g) transferring the single-cell suspension of (f) to an adherent substrate and expanding cell number in vitro to establish a population of purified adherent melanoma cancer stem cells comprising CD146+/CD271−, CD146+/CD271+, and CD146−/CD271+ cells.

27.-30. (canceled)

31. The method according to claim 26, wherein the population of purified adherent cancer stem cells comprises at least 20%, at least 30%, at least 40%, at least 50%, at least 60%, at least 70%, at least 80%, at least 90%, at least 95%, or at least 98% cells expressing CD146.

32. The method according to claim 26, wherein the population of purified adherent cancer stem cells comprises at least 20%, at least 30%, at least 40%, at least 50%, at least 60%, at least 70%, at least 80%, at least 90%, at least 95%, or at least 98% cells expressing CD271.

33. The method according to claim 26, wherein the population of purified adherent cancer stem cells comprises at least 20%, at least 30%, at least 40%, at least 50%, at least 60%, at least 70%, at least 80%, at least 90%, at least 95%, or at least 98% cells co-expressing CD146 and CD271.

34. The method according to claim 26, wherein the population of purified adherent cancer stem cells comprises 3.04% CD146+/CD271− cells, 93.39% CD146+/CD271+ cells, 0.55% CD146−/CD271− cells, and 3.02% CD146−/CD271+ cells.

35. The method according to claim 26, wherein the population of purified adherent cancer stem cells comprises 2.93% CD146+/CD271− cells, 92.45% CD146+/CD271+ cells, 1.25% CD146−/CD271− cells, and 3.37% CD146−/CD271+ cells.

36. The method according to claim 26, wherein the population of purified adherent cancer stem cells comprises 14.52% CD146+/CD271− cells, 77.84% CD146+/CD271+ cells, 5.7% CD146−/CD271− cells, and 2.44% CD146−/CD271+ cells.

37. The method according to claim 26, wherein the population of purified adherent cancer stem cells has: (i) a down-regulated immunosuppressive molecule; (ii) an up-regulated MHC-II; or (iii) a down-regulated immunosuppressive molecule and an up-regulated MHC-II, as compared with expression that is detectable in the single-cell suspension of (c).

38. The method according to claim 37, wherein the immunosuppressive molecule is at least one of indoleamine-pyrrole-2,3-dioxygenase, tumor growth factor-beta, and interleukin-10 (IL-10).

39. The method according to claim 37, wherein expression of the down-regulated immunosuppressive molecule is at a level less than 80%, less than 70%, less than 60%, less than 50%, less than 40%, less than 30%, less than 20%, or less than 10% of expression that is detectable in the single-cell suspension of (c).

40. The method according to claim 37, wherein expression of the down-regulated immunosuppressive molecule is to a level that is 80% or lower as compared with expression that is detectable in the single-cell suspension of (c).

41. The method according to claim 26, wherein the population of purified adherent cancer stem cells is enriched for CD146+/CD271−, CD146+/CD271+, and CD146−/CD271+ cells.

42. Use of the population of purified adherent cancer stem cells according to claim 26 for the preparation of a vaccine comprising dendritic cells loaded with the purified adherent cancer stem cells, wherein the dendritic cells and the cancer stem cells are from the same human subject.

43. The use according to claim 42, wherein the vaccine further comprises an immune adjuvant.

44. The use according to claim 43, wherein the immune adjuvant is selected from the group consisting of a toll-like receptor (TLR) agonist, a CD40 agonist, a cytokine and a combination thereof.

45. The use according to claim 44, wherein the toll-like receptor (TLR) agonist is selected from the group consisting of CpG-oligonucleotide (TLR9), imiquimod (TLR7), poly(I:C) (TLR3), glucopyranosyl lipid A (TLR4) murein (TLR2) and flagellin (TLR5).

46. The use according to claim 44, wherein the CD40 agonist is CD40-ligand.

47. The use according to claim 44, wherein the cytokine is selected from the group consisting of interferon-gamma and prostaglandin E2.

48. The method according to claim 26, wherein the cell culture media is Neuroblast stem cell media.
